SEQUENCE LISTING (13 sequences)

SEQ ID NO: 1 - DNA, 20 bp, Artificial Sequence
  Synthetic Sequence
ggccaagcacgagaacactc

SEQ ID NO: 2 - DNA, 20 bp, Artificial Sequence
  Synthetic Sequence
cgagaccacccggtggaggt

SEQ ID NO: 3 - DNA, 20 bp, Artificial Sequence
  Synthetic Sequence
cagctctccggccaaaggtt

SEQ ID NO: 4 - DNA, 25 bp, Artificial Sequence
  Synthetic Sequence
cagtttttcccaggaccttccgaac

SEQ ID NO: 5 - DNA, 24 bp, Artificial Sequence
  Synthetic Sequence
agtttttcccaggaccttccgaac

SEQ ID NO: 6 - DNA, 22 bp, Artificial Sequence
  Synthetic Sequence
gtttttccaggaccttccgaac

SEQ ID NO: 7 - DNA, 19 bp, Artificial Sequence
  Synthetic Sequence
agctgcgaagacttcacca

SEQ ID NO: 8 - DNA, 37 bp, Artificial Sequence
  Synthetic Sequence
agcagctccgagaccacccggtggaggtgggtgacaa

SEQ ID NO: 9 - DNA, 14 bp, Artificial Sequence
  Synthetic Sequence
agcagctccgagcg

SEQ ID NO: 10 - DNA, 30 bp, Artificial Sequence
  Synthetic Sequence
cagtatctggccaagcacgagaacatccac

SEQ ID NO: 11 - DNA, 29 bp, Artificial Sequence
  Synthetic Sequence
agcagctccgagaccacccggtggatgtt

SEQ ID NO: 12 - DNA, 25 bp, Artificial Sequence
  Synthetic Sequence
cagtttttcccaggaccttccgaac

SEQ ID NO: 13 - DNA, 66 bp, Artificial Sequence
  Synthetic Sequence
agctgcgaagacttcaggtcccgaggttctcgtgcttggccagatactgaagcacctcccgggcac

(19) United States
(12) Patent Application Publication
(10) Pub. No.: US 2024/0018474 A1
(43) Pub. Date: Jan. 18, 2024

(54) MODULATING BHLHE40 IN THE DIFFERENTIATION OF TYPE 1 REGULATORY T CELLS AND CONTROLLING T CELL EXHAUSTION

(71) Applicant: The Board of Trustees of the Leland Stanford Junior University, Stanford, CA (US)

(72) Inventors: Maria Grazia Roncarolo, Menlo Park (CA); Molly Kathryn Javier Uyeda, Stanford, CA (US); Robert Freeborn, Redwood City, CA (US)

(21) Appl. No.: 18/036,111

(22) PCT Filed: Nov. 12, 2021
(86) PCT No.: PCT/US2021/059137

§ 371 Date: May 9, 2023

Related U.S. Application Data
(60) Provisional application No. 63/113,369, filed on Nov. 13, 2020.

(51) Int. Cl. C12N 5/0783; A61K 35/17

(52) U.S. Cl. CPC C12N 5/0637; A61K 35/17; C12N 2506/11; C12N 2510/00; C12N 2501/231; C12N 2502/1121

(57) ABSTRACT

Methods and cell compositions are provided in which expression of BHLHE40 is modulated to direct T cell phenotype and function.

### CROSS REFERENCE TO RELATED APPLICATION

The present application claims the benefit and priority to PCT Application No. PCT/US2021/059137, filed Nov. 12, 2021, which claims priority to U.S. Provisional Patent Application No. 63/113,369, filed Nov. 13, 2020, which application are incorporated herein by reference in their entirety.

### INCORPORATION BY REFERENCE OF SEQUENCE LISTING

A Sequence Listing is provided herewith as a Sequence Listing text, STAN-1809_SeqListing_ST25, created on May 9, 2023, and having a size of 2,940 bytes. The contents of the Sequence Listing text are incorporated herein by reference in their entirety.

### BACKGROUND

T regulatory (Treg) cells are multifaceted-immunomodulatory cells that are composed of two subgroups: thymus- (tTreg) and peripherally-derived. Type 1 regulatory T (Tr1) cells are a peripherally-derived subset that is induced under tolerogenic conditions in an antigen-specific manner. Tr1 cells regulate the function of other CD4+ and CD8+ T cells directly and indirectly through antigen presenting cells predominately by secreting high levels of the pleiotropic cytokine IL-10. Unlike other IL-10 producing T helper cells, such as Th2 and Th17, Tr1 cells have a unique cytokine profile: high IL-10 and TGF-β, low IL-2, no IL-4 and IL-17, and variable amounts of IFNγ. Besides IL-10 production, mouse and human Tr1 cells can be identified by the co-expression of LAG3 and CD49b. With precise gating, Tr1 cells represent approximately 1-5% of the memory CD4+ T cells in the peripheral blood of human. This low Tr1 frequency in the periphery has posed challenges for studying them as compared to their FOXP3+ Treg counterparts, which are more abundant and constitutively express high levels of FOXP3, a master transcription factor.

While a master transcription factor has not been identified in Tr1 cells, the dominant role of IL-10 in Tr1 cell differentiation and function suggests that the molecular machinery controlling Tr1 cell identity is IL-10-centric. The IL-10 locus and promoter has been annotated with many known transcription factor binding sites utilized by many immune and non-immune cell types. The prevailing theory of IL-10 regulation in mouse CD4+ T subsets is that BHLHE40 inhibits IL-10 while c-Maf activates IL-10. While a genomic repressor and enhancer has been identified near the IL-10 transcriptional start site for BHLHE40 and c-MAF respectively, their expression is inversely correlated in c-MAF and BHLHE40 single knockout models, suggesting both direct and indirect IL-10 mediated regulation.

In mouse models, BHLHE40 regulates the immune response to resolve Toxoplasma gondii and Mycobacterium tuberculosis infections by downregulating IL-10 and upregulating IFNγ. BHLHE40 function expands further than just IL-10 regulation. In response to Heligmosomoides polygyrus, it ensures expression of Th2 cytokines such as IL-4 and IL-5; whereas in steady state, it prevents systemic autoimmunity and maintains Treg frequencies. The multifunctional role of BHLHE40 in different CD4+ T cell subsets, Tr1 cells especially, has yet to be delineated in human cells.

Exhausted T cells are T cells that become dysfunctional after a period of time and are potentially responsible for the lack of efficacy of T cell based therapies and for persistence in vivo. Weeks after infusion, sometimes a cell product is no longer detectable in the peripheral blood, potentially due to exhaustion or lack of engraftment. Additionally, most T cells become exhausted when they cannot properly differentiate into effector memory T cells, thereby disrupting their persistence in the body.

Specific transcription factors are upregulated during the naive to memory transition and certain transcription factors remain highly expressed in memory cells. These transcription factors control multiple cell processes, but most importantly for CD4 T cells, they control the cell's ability to produce cytokines. Some exhausted T cells will not produce the proper combination of cytokines—thereby affecting their polyfunctionality, the incorrect cytokine, or lower amounts of a cytokine. By modulating the expression of transcription factors, the functionality of memory T cells may be maintained, potentially reduces their propensity for exhaustion.

### SUMMARY

Methods and cell compositions are provided in which expression of BHLHE40 is modulated in a T cell population to direct the T cell phenotype and function. In some embodiments, methods are provided wherein naïve T cells are downregulated for BHLHE40 expression, thereby enhancing the cell's ability to differentiate into Tr1 cells. In some such embodiments, engineered cells are transiently down-regulated for BHLHE40 expression, e.g. with anti-sense RNA, RNAi, small molecule down-regulation, and the like. In other embodiments, engineered cells are genetically modified to knock out BHLHE40, including without limitation CRISPR deletions, for example in exon 5, which contains a domain that is required for DNA binding. The cells may be further differentiated in vitro to generate a population of engineered Tr1 cells.

It is shown herein that memory T cells and specifically human Type 1 regulatory T cells (Tr1 cells) express the transcription factor BHLHE40 at high levels. BHLHE40 expression allows efficient expression of certain cytokines, including without limitation IFNγ, IL4, and IL-2. In populations of total CD4⁺ T cells and in populations of naive T cells, the lack of BHLHE40 can lead to a significant reduction in IFNγ and IL-2 expression. However, it is also shown that differentiation of Tr1 cells from a population of naïve T cells is enhanced by reducing BHLHE40 expression.

In some embodiments, T cells down-regulated for BHLHE40, e.g. naïve T cells, are differentiated to Tr1 cells in vitro in a mixed lymphocyte reaction that contains: allogenic-tolerogenic dendritic cells, referred to as DC-10, CD4+ T cells, and IL-10, which induces allo-antigen specific anergic T cells, referred to herein as Tallo10. The resulting Tr1 cells can be characterized as LAG3⁺CD49b⁺. In some embodiments the differentiated cell population is selected for expression of LAG3 and CD49b, e.g. by flow cytometry, affinity purification methods, etc. to provide for a purified Tallo10 population.

In other embodiments, a T cell, e.g. a CAR T cell, is engineered to over-express BHLHE40, which cells are reduced in their tendency to exhaustion.

In some embodiments, an engineered cell is provided, in which the engineered cell has been treated to regulate BHLHE40 expression, e.g. by transient down-regulation, introduction of a coding sequence to upregulate BHLHE40, genetically modified to knock out BHLHE40, and the like. In some embodiments the cell is a human cell. In some embodiments, the cell is a human T cell, including without limitation naïve CD8⁺ T cells, cytotoxic CD8⁺ T cells, naïve CD4⁺ T cells, helper T cells, e.g. T_{H}1, T_{H}2, T_{H}9, T_{H}11, T_{H}22, T_{FH}; regulatory T cells, e.g. T_{R}1, natural T_{Reg}, inducible T_{Reg}; memory T cells, e.g. central memory T cells, effector memory T cells, NKT cells, αβ T cells, γδ T cells and engineered variants of such T cells including CAR T cells; Tallo10 cells, etc. The engineered cell can be provided in a unit dose for therapy, and can be allogeneic, autologous, etc. with respect to an intended recipient.

In some embodiments, a vector comprising a polynucleotide BHLHE40 coding sequence is provided, where the BHLHE40 sequence is operably linked to a promoter active in the desired T cell. An active promoter may be constitutively active or may be regulated. In some embodiments, the vector comprises a polynucleotide coding sequence that downregulates BHLHE40, e.g. encoding guide RNA, a knockout construct, RNAi construct, antisense construct, etc. Various vectors are known in the art and can be used for this purpose, e.g. replication competent, replication deficient or conditionally replicating viral vectors, plasmid vectors, minicircle vectors. In some embodiments, the vector maybe integrated into the target cell genome or can be episomally maintained.

In some embodiments a therapeutic method is provided, the method comprising introducing into a subject in need thereof a therapeutically effective quantity of an engineered cell population, in which the engineered cell has been treated to regulate BHLHE40 expression. The cell population may be engineered ex vivo, and may be autologous or allogeneic with respect to the subject. In some embodiments the engineered cell is a CAR T cell, e.g. by transient down-regulation, introduction of a coding sequence to upregulate BHLHE40, genetically modified to knock out BHLHE40, and the like. In some embodiments the engineered cell is a Tr1 cell. In some embodiments the engineered cell is a Tallo10 cell.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1A-FIG. 1D. RNA sequencing identifies BHLHE40 in Tr1 cells. FIG. 1A) Sorting strategy for ex vivo human Tr1 cells. Healthy donor PBMC were gated for CD3+/CD4+/CD45RA−. Tr1 cells were additionally gated and sorted for LAG3+/CD49b+, indicated with a red bounding box, while non-Tr1 T memory cells were sorted for LAG3−/CD49b−, indicated with a black bounding box. FIG. 1B) Volcano plot of differentially expressed genes between Tr1 and non-Tr1. Dots indicate individual genes, with genes having an adjusted p value<0.05 indicated in red and with genes with also an abs(log2(fold change))>1 in orange. FIG. 1C) Differentially expressed transcription factors between Tr1 and non-Tr1 Tmemory with an adjusted p value<0.05 plotted by rows and individual CD4+ T cell in columns. Fill color is z-scaled per gene. FIG. 1D) Normalized counts (variance stabilized normalized counts) of BHLHE40 gene expression between Tr1 and non-Tr1. *=adjusted p value<0.05 after Benjamini-Hochberg multiple testing correction. N=3, biological duplicates for Tr1 and Tmem. Tmem=non-Tr1 T memory.

FIG. 2A-FIG. 2F. BHLHE40 knockout leads to reduction in IFNγ and IL-4 expression. FIG. 2A) BHLHE40 knockout strategy. 3 sgRNA (red) were designed to simultaneous target the N′ terminus exon 5 of BHLHE40. Protein domains (black) and exons (grey) are annotated. FIG. 2B) High knockout efficiency in total CD4+ T cells. Indels % were calculated at day 3 after nucleofection. FIG. 2C) Timeline of generating BHLHE40 knockout cells. CD4+ T cells from healthy donors were thawed overnight in IL-2 and nucleofected the following morning using three different sgRNA targeting exon 5. gDNA was collected 2 days later and the remaining cells were stimulated with CD3/CD2/CD28 for 3 days and further expanded with IL-2 for 11 days. FIG. 2D) Cytokine Secretion. T cells were stimulated with plate-bound CD3 and soluble CD28 and supernatant was collected at 24 h (IL-2) and 48 h (IFNg, IL-4, IL-10) to measure cytokines by ELISA. FIG. 2E) In vitro expansion. After 3 rounds of stimulation and expansion, cells were collected and counted with an automated cell counter. FIG. 2F) Representative flow cytometry histogram of CFSE dilution 3 days post CD3/CD28 Dynabead stimulation. Gated on live singlets. (left) Cumulative CFSE proliferation data of all samples (right). n=7, *=p value<0.05, Wilcoxon matched-pairs signed rank test.

FIG. 3A-FIG. 3D. BHLHE40 deficiency increases IL-10 in naïve CD4+ T cells. FIG. 3A) High knockout efficiency in naïve CD4+ T cells. Indels % were calculated at day 3 after nucleofection. FIG. 3B) Cytokine Secretion. T cells were stimulated with plate-bound CD3 and soluble CD28 and supernatant was collected at 24 h (IL-2) and 48 h (IFNg, IL-4, IL-10) to measure cytokines by ELISA. FIG. 3C) In vitro expansion of mock and sgBHLHE40 treated naïve T cells. Cells were collected and counted with an automated cell counter after 2 rounds of expansion. FIG. 3D) Representative flow cytometry histogram of CFSE dilution 3 days post CD3/CD28 Dynabead stimulation. Gated on live singlets. (left) Cumulative CFSE proliferation data of all samples (right). n=10, *=p value<0.05, **=p value<0.01, Wilcoxon matched-pairs signed rank test.

FIG. 4A-FIG. 4F. BHLHE40 is not required for induction of allo-antigen specific Tr1 cells. FIG. 4A) CRIPSR-Cas9 editing and Tr1 induction timeline. CD4+ T cells were edited at the BHLHE40 locus and then plated with either DC-10 or matDC for 10 days from an allogenic donor. At day 10, recovered cells were characterized. FIG. 4B) BHLEH40 Editing Efficiency. gDNA of edited T cells was collected 2 days after editing and checked for indels. FIG. 4C) Tr1 induction efficiency is inhibited. At day 10, cells were analyzed by flow cytometry for the expression of Tr1 specific markers (CD3+/CD4+/CD45RA−/LAG3+/CD49b+). (left) A representative plot of Tallo10 cells gated on CD3+/CD4+/CD45RA− (right) summarized data. FIG. 4D) T cells do not require BHLHE40 to become anergic towards allo-antigen. CFSE labeled mock and BHLHE40 edited Tallo and Tallo10 T cells were co-cultured with matDC for 3 days and dye dilution was assessed by flow cytometry. Anergy was calculated using the following equation (% proliferated Tallo mock−% proliferated Tallo10 mock or sgBHLHE40)/% proliferated Tallo mock. FIG. 4E) IFNγ but not IL-10 is reduced in sgBHLHE40 Tallo10 cultures. Supernatant from wells plated for anergy as described in D) was collected at day 3 and the indicated cytokines were quantified by ELISA. Bars represent the mean and the errors bars represent standard deviation. FIG. 4F) Cells were co-cultured with 1 CD3/CD28 Dynabead per 20 cells for 72 hours and supernatant was collected to measure IL-10 via ELISA.

FIG. 5A-FIG. 5F. Overexpression of BHLHE40 induces co-expression of CD49b and LAG3 in naïve CD4+ T cells. FIG. 5(A) Validation of a dox inducible system for transgene overexpression in CD4+ T cells. pCW57.1 construct used for expressing GFP or BHLHE40 (top). GFP expression in CD4+ T cells is induced after addition of dox. Transgene expression is maintained during activation. Dox was incubated for 4 hours and cells were immediately collected and checked for GFP expression by flow cytometry (Bottom-left). BHLHE40 overexpression confirmed by qPCR. GFP and BHLHE40 transduced CD4+ T cells were incubated with dox for 4 hours and RNA was collected. BHLHE40 RNA was measured using qPCR Taqman probes along with housekeeping genes: RPLPO, GAPDH. n=3 (Bottom-right). FIG. 5(B) Timeline of dox-inducible BHLHE40 in naïve CD4+ T cells. CD4+ naïve T cells were cultured with IL-10 and then expanded on allo-feeders with IL-2 for 14 days before being characterized. FIG. 5(C-D) Surface phenotype of transduced cells after 2 rounds of allo-feeder expansion. Gated on live, CD3+, CD4+ cells (C) CD49b and LAG3 expression. Representative plots of CD49b and LAG3 in one donor (Left). Summarized data of CD49b and LAG3 expression on BHLHE40 overexpressing cells compared to GFP expressing cells. (Right). n=6. (D) Co-expression of LAG3 and PD1. Representative plots of PD1 and LAG3 in one donor (Left). Summarized data of PD1 and LAG3 expression on BHLHE40 overexpressing cells compared to GFP expressing cells (Right). n=6. (E-F) Increased Th1 phenotypic cytokines in BHLHE40 overexpressing cells. FIG. 5(E) Increased intracellular IFN-γ+/IL4− cells in BHLHE40 overexpressing cells. Intracellular cytokine staining of IFN-γ and IL-4 in BHLHE40 overexpressing cells after 5 hours of PMA/I and Brefeldin A. n=6. Ratio paired t-test. FIG. 5(F) Increased secretion of IFN-γ. Cells are stimulated for 48 hours with plate-bound αCD3 and soluble αCD28.Cytokines in the supernatant were quantified by ELISA. IFNγ/IL-4 ratios (Left). Absolute IL-10 concentrations (Right). n=5, Ratio paired t test. PMA/I=phorbol-myristate-acetate/ionomycin, Allo-feeders=allogenic-PBMC feeders, Dox=doxycycline, dpf=days post feeder, puro=puromycin. *=p value<0.05.

FIG. 6A-FIG. 6C. Validation of BHLHE40 knockout strategy. FIG. 6(A) (Top) Representative examples of genomic editing results using 3 multiplex guides against BHLHE40 using the tool: Inference of CRISPR Edits (ICE). (Bottom) ICE discordance data of sgBHLHE40-edited samples in green and mock-treated samples in orange. FIG. 6(B) (Left) Protein Simple Wes generated images of separated protein lysates. Lanes 1 and 2 were run with HSP90 antibody and lanes 3 and 4 were run with BHLHE40 polyclonal antibody with the following parameters: 0.5 μg protein/lane, separation time 55 mins, exposure time 5 s. (Right) Summarize data of genomic and protein knockouts (KO). Protein knockout was calculated by dividing the normalized area under the curve (BHLHE40/HSP90) for sgBHLHE40-edited samples by mock-treated samples. n=7

FIG. 7A-FIG. 7B. Method for testing if knocking out BHLHE40 in naïve CD4+ T cells and differentiating them into Tr1 in vitro is more efficient than wild type. FIG. 7A) Tr1 induction follows previously described protocol first validated in Bacchetta et al. 2014 and further improved in NCT03198234 in which allogenic CD14+ cells are differentiated into mature DC (matDC), immature DC (iDC), or tolerogenic DC (DC-10), or DC-10 with 10 ng/ml of IL-10 as described in NCT03198234. FIG. 7B) Allogenic naïve CD4+ T cells isolated from umbilical cord blood are isolated using magnetic cell separation tools and BHLHE40 is genomically knocked out by nucleofecting RNP Cas9 multiplexed with 3 sgRNA as described in the METHODS. Mock indicates cells that are nucleofected without any RNP. T cells and the indicated allogenic DC are co-cultured for 10 days (day 7-day 17).

FIG. 8. Naïve BHLHE40 KO cells differentiate into LAG3+/CD49b+ Tr1 cells more efficiently. At day 17, cells were collected and stained with CD3, CD4, CD45RA, LAG3, CD49b, and Ghost510 (viability dye). After staining, cells were assessed for surface antigen expression on a flow cytometer. Cells were gated on lymphocytes/singlets/live/CD3+/CD4+/CD45RA−. N=1.

FIG. 9. BHLHE40 KO cultured with iDC and DC10 are more hyporesponsive to allogenic antigen. At day 17 cells were collected and stained with CFSE and plated with matDC for 3 days; 1 matDC for every 10 T cells. After 3 days cells were stained with CD3, CD4, and Dapi (viability). Cells were assessed on a flow cytometer. Histograms are pregated on Dapi−/CD3+/CD4+/ and the CFSE dilution is indicated, which represent the percentage of cells that are responding to allogenic antigen over 3 days.

FIG. 10. All cells were proliferative when stimulated polyclonally. At day 17 cells were collected and stained with CFSE and plated with CD3/CD28 dynabeads, 1 CD3/CD28 Dynabead per 20 cells. After 3 days cells were stained with CD3, CD4, and Dapi (viability). Cells were assessed on a flow cytometer. Histograms are pregated on Dapi−/CD3+/CD4+/ and the CFSE dilution is indicated, which represent the percentage of cells that are responding to polyclonal stimulation over 3 days.

FIG. 11. Cytokine gene expression in naïve CD4+ T cells. Following 2 rounds of expansion with allogeneic feeder cells, mock-treated or sgBHLHE40-edited naïve CD4+ T cells were stimulated with aCD3/aCD28 Dynabeads. RNA was collected 6 hours post-stimulation and was reverse transcribed. IFN-γ, IL-4, and IL-2 were quantified in the resulting cDNA using qPCR Taqman probes and normalized to the housekeeping gene, RPLPO. n=6. ns=not significant. Wilcoxon matched-pairs signed rank test.

FIG. 12. Cytokine gene expression in total CD4+ T cells. Following 2 rounds of expansion with allogeneic feeder cells, mock-treated or sgBHLHE40-edited total CD4+ T cells were stimulated with aCD3/aCD28 Dynabeads. RNA was collected 6 hours post-stimulation and was reverse transcribed. IFN-γ, IL-4, and IL-2 were quantified in the resulting cDNA using qPCR Taqman probes and normalized to the housekeeping gene, RPLPO. n=6. ns=not significant. Wilcoxon matched-pairs signed rank test.

FIG. 13. CD4⁺ naïve T cells were cultured with IL-10 and then expanded on allo-feeders with IL-2 for 14 days before being characterized. Surface phenotype of transduced cells after 2 rounds of allo-feeder expansion. Gated on live, CD3⁺, CD4⁺ cells. CD49b and LAG3 expression. Independent donors are shown in columns. The first two columns and the last two columns are donors from two independent overexpression experiments with flow cytometry data acquired on separate days.

FIG. 14 depicts an example of an expression construct for BHLHE40.

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “sequence identity,” as used herein in reference to polypeptide or DNA sequences, refers to the subunit sequence identity between two molecules. When a subunit position in both of the molecules is occupied by the same monomeric subunit (e.g., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990).

By “protein variant” or “variant protein” or “variant polypeptide” herein is meant a protein that differs from a wild-type protein by virtue of at least one amino acid modification. The parent polypeptide may be a naturally occurring or wild-type (WT) polypeptide, or may be a modified version of a WT polypeptide. Variant polypeptide may refer to the polypeptide itself, a composition comprising the polypeptide, or the amino sequence that encodes it. Preferably, the variant polypeptide has at least one amino acid modification compared to the parent polypeptide, e.g. from about one to about ten amino acid modifications, and preferably from about one to about five amino acid modifications compared to the parent.

By “parent polypeptide”, “parent protein”, “precursor polypeptide”, or “precursor protein” as used herein is meant an unmodified polypeptide that is subsequently modified to generate a variant. A parent polypeptide may be a wild-type (or native) polypeptide, or a variant or engineered version of a wild-type polypeptide. Parent polypeptide may refer to the polypeptide itself, compositions that comprise the parent polypeptide, or the amino acid sequence that encodes it.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. “Amino acid analogs” refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acid modifications may include amino acid substitutions, deletions and insertions, particularly amino acid substitutions. Variant proteins may also include conservative modifications and substitutions at other positions of the cytokine and/or receptor (e.g., positions other than those involved in the affinity engineering). Such conservative substitutions include those described by Dayhoff in The Atlas of Protein Sequence and Structure 5 (1978), and by Argos in EMBO J., 8:779-785 (1989). For example, amino acids belonging to one of the following groups represent conservative changes: Group I: Ala, Pro, Gly, Gln, Asn, Ser, Thr; Group II: Cys, Ser, Tyr, Thr; Group III: Val, Ile, Leu, Met, Ala, Phe; Group IV: Lys, Arg, His; Group V: Phe, Tyr, Trp, His; and Group VI: Asp, Glu. Further, amino acid substitutions with a designated amino acid may be replaced with a conservative change.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure. A “separated” compound refers to a compound that is removed from at least 90% of at least one component of a sample from which the compound was obtained. Any compound described herein can be provided as an isolated or separated compound.

The terms “subject,” “individual,” and “patient” are used interchangeably herein to refer to a mammal being assessed for treatment and/or being treated. In some embodiments, the mammal is a human. The terms “subject,” “individual,” and “patient” encompass, without limitation, individuals having a disease. Subjects may be human, but also include other mammals, particularly those mammals useful as laboratory models for human disease, e.g., mice, rats, etc.

The term “sample” with reference to a patient encompasses blood and other liquid samples of biological origin, solid tissue samples such as a biopsy specimen or tissue cultures or cells derived therefrom and the progeny thereof. The term also encompasses samples that have been manipulated in any way after their procurement, such as by treatment with reagents; washed; or enrichment for certain cell populations, such as diseased cells. The definition also includes samples that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's diseased cell, e.g., a sample comprising polynucleotides and/or polypeptides that is obtained from a patient's diseased cell (e.g., a cell lysate or other cell extract comprising polynucleotides and/or polypeptides); and a sample comprising diseased cells from a patient. A biological sample comprising a diseased cell from a patient can also include non-diseased cells.

The term “diagnosis” is used herein to refer to the identification of a molecular or pathological state, disease or condition in a subject, individual, or patient.

The term “prognosis” is used herein to refer to the prediction of the likelihood of death or disease progression, including recurrence, spread, and drug resistance, in a subject, individual, or patient. The term “prediction” is used herein to refer to the act of foretelling or estimating, based on observation, experience, or scientific reasoning, the likelihood of a subject, individual, or patient experiencing a particular event or clinical outcome. In one example, a physician may attempt to predict the likelihood that a patient will survive.

As used herein, the terms “treatment,” “treating,” and the like, refer to administering an agent, or carrying out a procedure, for the purposes of obtaining an effect on or in a subject, individual, or patient. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of cancer in a mammal, particularly in a human, and includes: (a) inhibiting the disease, i.e., arresting its development; and (b) relieving the disease or its symptoms, i.e., causing regression of the disease or its symptoms.

Treating may refer to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of engineered cells to prevent or delay, to alleviate, or to arrest or inhibit development of the symptoms or conditions associated with disease or other diseases. The term “therapeutic effect” refers to the reduction, elimination, or prevention of the disease, symptoms of the disease, or side effects of the disease in the subject.

As used herein, a “therapeutically effective amount” refers to that amount of the therapeutic agent sufficient to treat or manage a disease or disorder. A therapeutically effective amount may refer to the amount of therapeutic agent sufficient to delay or minimize the onset of disease, e.g., to delay or minimize the growth and spread of cancer. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

“In combination with”, “combination therapy” and “combination products” refer, in certain embodiments, to the concurrent administration to a patient of the engineered proteins and cells described herein in combination with additional therapies, e.g. surgery, radiation, chemotherapy, and the like. When administered in combination, each component can be administered at the same time or sequentially in any order at different points in time. Thus, each component can be administered separately but sufficiently closely in time so as to provide the desired therapeutic effect.

“Concomitant administration” means administration of one or more components, such as engineered proteins and cells, known therapeutic agents, etc. at such time that the combination will have a therapeutic effect. Such concomitant administration may involve concurrent (i.e. at the same time), prior, or subsequent administration of components. A person of ordinary skill in the art would have no difficulty determining the appropriate timing, sequence and dosages of administration.

The use of the term “in combination” does not restrict the order in which agents or cells are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

BHLHE40. Bhlhe40 (also known as Dec1, Stra13, Sharp2 or Bhlhb2) belongs to a family of basic helix-loop-helix transcriptional regulators sharing structural features including a basic DNA-binding domain, a helix-loop-helix domain mediating dimerization and a protein-protein interaction ‘Orange domain’. Members of this family are known to respond to environmental stimuli and regulate several physiological processes in diverse cell types, including the cell cycle, apoptosis and differentiation via their actions as both transcriptional activators and repressors. Bhlhe40 is expressed in T cells upon T-cell receptor (TCR) stimulation.

Genetic sequences for BHLHE40 can be accessed at public databases, including for example Genbank, where the human mRNA and protein sequences are available as NM_003670 and NP_003661, respectively; and the mouse sequences as NM_011498 and NP_035628. A sequence can be substantially similar to a known sequence, e.g. having at least about 99% sequence identity, at least about 98%, 97%, 95%, 90% 85% identity.

### Cell Compositions

In some embodiments a cell composition is provided. The cell can be provided in a unit dose for therapy, and can be allogeneic, autologous, etc. with respect to an intended recipient. Methods may include a step of obtaining desired cells, e.g., T cells, etc., which may be isolated from a biological sample, or may be derived in vitro from a source of progenitor cells. The cells are engineered as described herein, which may be performed in any suitable culture medium. For example, cells may be collected from a patient, modified ex vivo, and reintroduced into the subject. The cells collected from the subject may be collected from any convenient and appropriate source, including e.g., peripheral blood (e.g., the subject's peripheral blood), a biopsy (e.g., a biopsy from the subject), and the like.

Engineered cells can be provided in pharmaceutical compositions suitable for therapeutic use, e.g. for human treatment. Therapeutic formulations comprising such cells can be frozen, or prepared for administration with physiologically acceptable carriers, excipients or stabilizers (Remington's Pharmaceutical Sciences 16th edition, Osol, A. Ed. (1980)), in the form of aqueous solutions. The cells will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The cells can be administered by any suitable means, usually parenteral. Parenteral infusions include intramuscular, intravenous (bolus or slow drip), intraarterial, intraperitoneal, intrathecal or subcutaneous administration.

Type 1 regulatory T (Tr1) cells are an inducible subset of regulatory T cells that play a pivotal role in promoting and maintaining tolerance. The main mechanisms by which Tr1 cells control immune responses are the secretion of high levels of IL-10, and the killing of myeloid myeloid cells through the release of Granzyme B. Tr1 cells secrete high levels of IL-10 and minimal amounts of IL-4 and IL-17, which distinguish them from Th2 and Th17. Furthermore, Tr1 cells secrete low levels of IL-2, and depending on the local cytokine milieu can produce variable levels of IFN-γ. Similar to other T cell subsets, Tr1 cells can transiently express FOXP3 upon activation; however, in Tr1 cells FOXP3 expression is not constitutive and never reaches the high levels characteristic of CD25+ Foxp3+ regulatory T cells.

In some embodiments a method to generate allo-specific Tr1 cells, cultures in vitro with IL-10 a population of naïve T cells or total CD4⁺ T cells treated to down-regulate BHLHE40. The population of naïve T cells can be obtained from a patient in which allo-specific tolerance is desired, e.g. a donor of an allogeneic graft. A population of naïve T cells can be selected for the phenotype of CD45RA⁺CD4⁺, and may be obtained from cord blood, peripheral blood, etc.

The method to generate allo-specific Tr1 cells can utilize a Tallo10 differentiation protocol, for example as disclosed in clinical trial ID NCT03198234. Such a method differentiates Tallo10 cells by culture of CD4+ T cells in the presence of recombinant IL-10, and/or allogenic-tolerogenic dendritic cells, referred to as DC-10 (see, for example, Amodio and Gregori (2012) Transplant Res. 1: 14). DC-10 are differentiated from peripheral blood monocytes cultured in the presence of GM-CSF, IL-4, and IL-10. The resulting DC-10 cells are CD11c⁺ CD11b⁺ CD14⁺ CD16⁺ CD1a−, and display a mature myeloid phenotype, being CD83⁺, CD86⁺ and HLA-DR⁺. The cells secrete high amounts of IL-10; secrete IL-6, low levels of TNFα and no IL-12. DC-10 are phenotypically different and more potent than immature DCs or IL-10-modulated mature DC in promoting allo-specific Tr1 cells. The DC-10 cells may be be cultured from cells obtained from an individual to which allo-tolerance is desired, e.g. a transplant recipient.

The resulting Tallo10 cells are anergic, secrete significant levels of IL-10, and suppress T cell responses in vitro in an IL-10-dependent manner. Adoptive transfer of ex vivo induced alloantigen-specific Tr1 (Tallo10) cells has proven to be feasible and safe, and can be applied in allogeneic hematopoietic stem cell transplantation among other uses.

An alternative strategy for the induction of high numbers of human Tr1 cells is lentiviral-mediated gene transfer of human IL-10. Stable ectopic expression of IL-10 can efficiently generate homogeneous populations of Tr1-like cells. These cells display potent suppressive functions both in vitro and in vivo in xenogeneic graft versus host disease model, while preserving the graft versus leukemia effects.

In some embodiments, an engineered cell is provided, in which the cell has been modified by introduction of a expression vector or a vector for knocking out or reducing expression of BHLHE40. In other embodiments the engineered cell has been treated to transiently down-regulate expression of BHLHE40, e.g. by contacting the cell population with BHLHE40 anti-sense oligonucleotides, with BHLHE40 siRNA, etc.

An anti-BHLHE40 agent may be an shRNA or an antisense oligonucleotide (ODN). By RNAi agent is meant an agent that modulates expression by a RNA interference mechanism. The RNAi agents employed in one embodiment are small ribonucleic acid molecules (also referred to herein as interfering ribonucleic acids), i.e., oligoribonucleotides, that are present in duplex structures, e.g., two distinct oligoribonucleotides hybridized to each other or a single ribooligonucleotide that assumes a small hairpin formation to produce a duplex structure. By oligoribonucleotide is meant a ribonucleic acid that does not exceed about 100 nt in length, and typically does not exceed about 75 nt length, where the length in certain embodiments is less than about 70 nt. Where the RNA agent is a duplex structure of two distinct ribonucleic acids hybridized to each other, e.g., an siRNA, the length of the duplex structure typically ranges from about 15 to 30 bp, usually from about 15 to 29 bp, where lengths between about 20 and 29 bps, e.g., 21 bp, 22 bp, are of particular interest in certain embodiments. Where the RNA agent is a duplex structure of a single ribonucleic acid that is present in a hairpin formation, i.e., a shRNA, the length of the hybridized portion of the hairpin is typically the same as that provided above for the siRNA type of agent or longer by 4-8 nucleotides. The weight of the RNAi agents of this embodiment typically ranges from about 5,000 daltons to about 35,000 daltons, and in many embodiments is at least about 10,000 daltons and less than about 27,500 daltons, often less than about 25,000 daltons.

dsRNA can be prepared according to any of a number of methods that are known in the art, including in vitro and in vivo methods, as well as by synthetic chemistry approaches. Examples of such methods include, but are not limited to, the methods described by Sadher et al. (Biochem. Int. 14:1015, 1987); by Bhattacharyya (Nature 343:484, 1990); and by Livache, et al. (U.S. Pat. No. 5,795,715), each of which is incorporated herein by reference in its entirety. Single-stranded RNA can also be produced using a combination of enzymatic and organic synthesis or by total organic synthesis. The use of synthetic chemical methods enable one to introduce desired modified nucleotides or nucleotide analogs into the dsRNA. dsRNA can also be prepared in vivo according to a number of established methods (see, e.g., Sambrook, et al. (1989) Molecular Cloning: A Laboratory Manual, 2nd ed.; Transcription and Translation (B. D. Hames, and S. J. Higgins, Eds., 1984); DNA Cloning, volumes I and II (D. N. Glover, Ed., 1985); and Oligonucleotide Synthesis (M. J. Gait, Ed., 1984, each of which is incorporated herein by reference in its entirety).

In certain embodiments, instead of the RNAi agent being an interfering ribonucleic acid, e.g., an siRNA or shRNA as described above, the RNAi agent may encode an interfering ribonucleic acid, e.g., an shRNA, as described above. In other words, the RNAi agent may be a transcriptional template of the interfering ribonucleic acid. In these embodiments, the transcriptional template is typically a DNA that encodes the interfering ribonucleic acid. The DNA may be present in a vector, where a variety of different vectors are known in the art, e.g., a plasmid vector, a viral vector, etc.

Alternatively, an antisense sequence is complementary to the targeted RNA, and inhibits its expression. One or a combination of antisense molecules may be administered, where a combination may comprise multiple different sequences. Antisense molecules may be produced by expression of all or a part of the target RNA sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 25, usually not more than about 23-22 nucleotides in length, where the length is governed by efficiency of inhibition, specificity, including absence of cross-reactivity, and the like.

Anti-sense molecules of interest include antagomir RNAs, e.g. as described by Krutzfeldt et al. (2005) Nature 438:685-689, herein specifically incorporated by reference. Small interfering double-stranded RNAs (siRNAs) engineered with certain ‘drug-like’ properties such as chemical modifications for stability and cholesterol conjugation for delivery have been shown to achieve therapeutic silencing of an endogenous gene in vivo. To develop a pharmacological approach for silencing miRNAs in vivo, chemically modified, cholesterol-conjugated single-stranded RNA analogues complementary to miRNAs were developed, termed ‘antagomirs’. Antagomir RNAs may be synthesized using standard solid phase oligonucleotide synthesis protocols. The RNAs are conjugated to cholesterol, and may further have a phosphorothioate backbone at one or more positions.

In some embodiments an anti-BHLHE40 agent utilizes a class 2 CRISPR/Cas effector protein (or a nucleic encoding the protein), e.g., as targeted endonuclease to alter the genomic sequence at the BHLHE40 locus in a manner that decreases expression of BHLHE40, e.g. by deletion of a portion of the coding sequence. Exemplary guide RNAs include, without limitation: (SEQ ID NO:1) GGCCAAGCACGAGAACACTC, (SEQ ID NO:2) CGAGACCACCCGGTGGAGGT, (SEQ ID NO:3) CAGCTCTCCGGCCAAAGGTT. In class 2 CRISPR systems, the functions of the effector complex (e.g., the cleavage of target DNA) are carried out by a single protein (which can be referred to as a CRISPR/Cas effector protein)—where the natural protein is an endonuclease (e.g., see Zetsche et al, Cell. 2015 Oct. 22; 163(3):759-71; Makarova et al, Nat Rev Microbiol. 2015 November; 13(11):722-36; Shmakov et al., Mol Cell. 2015 Nov. 5; 60(3):385-97; and Shmakov et al., Nat Rev Microbiol. 2017 March; 15(3):169-182: “Diversity and evolution of class 2 CRISPR-Cas systems”). As such, the term “class 2 CRISPR/Cas protein” or “CRISPR/Cas effector protein” is used herein to encompass the effector protein from class 2 CRISPR systems—for example, type II CRISPR/Cas proteins (e.g., Cas9), type V CRISPR/Cas proteins (e.g., Cpf1/Cas12a, C2c1/Cas12b, C2C3/Cas12c), and type VI CRISPR/Cas proteins (e.g., C2c2/Cas13a, C2C7/Cas13c, C2c6/Cas13b). Class 2 CRISPR/Cas effector proteins include type II, type V, and type VI CRISPR/Cas proteins, but the term is also meant to encompass any class 2 CRISPR/Cas protein suitable for binding to a corresponding guide RNA and forming a ribonucleoprotein (RNP) complex.

A nucleic acid that binds to a class 2 CRISPR/Cas effector protein (e.g., a Cas9 protein; a type V or type VI CRISPR/Cas protein; a Cpf1 protein; etc.) and targets the complex to a specific location within a target nucleic acid is referred to herein as a “guide RNA” or “CRISPR/Cas guide nucleic acid” or “CRISPR/Cas guide RNA.” A guide RNA provides target specificity to the complex (the RNP complex) by including a targeting segment, which includes a guide sequence, which is a nucleotide sequence that is complementary to a sequence of a target nucleic acid.

Alternatively an engineered cell can be modified by introduction of a construct to over-express BHLHE40 by introduction of BHLHE40 coding sequences operably linked to a promoter active in T cells. The sequences can be introduced on a vector, e.g. a viral vector, etc., or can be introduced through genome editing. For example, a coding sequence may be introduced into a target cell using CRISPR technology. CRISPR/Cas9 system can be directly applied to human cells by transfection with a plasmid that encodes Cas9 and sgRNA. The viral delivery of CRISPR components has been extensively demonstrated using lentiviral and retroviral vectors. Gene editing with CRISPR encoded by non-integrating virus, such as adenovirus and adenovirus-associated virus (AAV), has also been reported. Recent discoveries of smaller Cas proteins have enabled and enhanced the combination of this technology with vectors that have gained increasing success for their safety profile and efficiency, such as AAV vectors.

The nucleic acid encoding a reprogaming factor is inserted into a vector for expression and/or integration. Many such vectors are available. The vector components generally include, but are not limited to, one or more of the following: an origin of replication, one or more marker genes, an enhancer element, a promoter, and a transcription termination sequence. Vectors include viral vectors, plasmid vectors, integrating vectors, and the like.

Expression vectors may contain a selection gene, also termed a selectable marker. This gene encodes a protein necessary for the survival or growth of transformed host cells grown in a selective culture medium or a truncated gene encoding a surface marker that allows for antibody based detection. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, or (d) enable surface antibody based detection for isolation via fluoresences activating cell sorting (FACS) or magnetic separation e.g. truncated forms of NGFR, EGFR, CD19.

Nucleic acids are “operably linked” when placed into a functional relationship with another nucleic acid sequence. For example, DNA for a signal sequence is operably linked to DNA for a polypeptide if it is expressed as a preprotein that signals the secretion of the polypeptide; a promoter or enhancer is operably linked to a coding sequence if it affects the transcription of the sequence; and a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous.

Expression vectors will contain a promoter that is recognized by the host organism and is operably linked to the coding sequence. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

Transcription from vectors in mammalian host cells may be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus LTR (such as murine stem cell virus), hepatitis-B virus and Simian Virus 40 (SV40), from heterologous mammalian promoters, e.g., the actin promoter, PGK (phosphoglycerate kinase), or an immunoglobulin promoter, or from heat-shock promoters, provided such promoters are compatible with the host cell systems. The early and late promoters of the SV40 virus are conveniently obtained as an SV40 restriction fragment that also contains the SV40 viral origin of replication.

Transcription by higher eukaryotes may be increased by inserting an enhancer sequence into the vector. Enhancers are cis-acting elements of DNA, usually about from 10 to 300 bp in length, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, α-fetoprotein, and insulin). Typically, however, one will use an enhancer from a eukaryotic virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence, but is preferably located at a site 5′ from the promoter.

Expression vectors for use in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

Suitable host cells for cloning a construct are the prokaryotic, yeast, or other eukaryotic cells described above. Examples of useful mammalian host cell lines are mouse L cells (L-M[K-], ATCC#CRL-2648), monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); Chinese hamster ovary cells/-DHFR (CHO); mouse Sertoli cells (TM4); monkey kidney cells (CV1 ATCC CCL 70); African green monkey kidney cells (VERO-76, ATCC CRL-1 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2).

Host cells, including CD4+ T cells, naïve T cells, stem cells, etc. can be transfected with the above-described expression vectors for construct expression. Cells may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression, and will be apparent to the ordinarily

In some embodiments an engineered cell is a T cell, including without limitation naïve CD8⁺ T cells, cytotoxic CD8⁺ T cells, naïve CD4⁺ T cells, helper T cells, e.g. T_{H}1, T_{H}2, T_{H}9, T_{H}11, T_{H}22, T_{FH}; regulatory T cells, e.g. T_{R}1, natural T_{Reg}, inducible T_{Reg}; memory T cells, e.g. central memory T cells, effector memory T cells, NK T cells, γδ T cells and variants of such T cells including CAR T cells, etc. In some embodiments the cell is genetically modified in an ex vivo procedure, prior to transfer into a subject, to introduce a sequence to upregulate or down-regulate expression. The engineered cell can be provided in a unit dose for therapy, and can be allogeneic, autologous, or xenogeneic with respect to an intended recipient, usually autologous.

T cells useful for engineering with the genetic agents disclosed above include naïve T cells, total CD4⁺ T cells, etc. T cells for treatment as described above can be collected from a subject or a donor, and may be separated from a mixture of cells by techniques that enrich for desired cells or may be engineered and cultured without separation. An appropriate solution may be used for dispersion or suspension of the cells. Such solution will generally be a sterile balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. Techniques for affinity separation may include magnetic separation, using antibody-coated magnetic beads, affinity chromatography, cytotoxic agents linked to a monoclonal antibody or used in conjunction with a monoclonal antibody, e.g., complement and cytotoxins, and “panning” with antibody attached to a solid matrix, e.g., a plate, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g., propidium iodide). Any technique may be employed which is not unduly detrimental to the viability of the selected cells. The affinity reagents may be specific receptors or ligands for the cell surface molecules indicated above. In addition to antibody reagents, peptide-MHC antigen and T cell receptor pairs may be used; peptide ligands and receptor; effector and receptor molecules, and the like.

The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum (FCS). The collected and optionally enriched cell population may be used immediately for genetic modification, or may be frozen at liquid nitrogen temperatures and stored, being thawed and capable of being reused. The cells will usually be stored in 10% DMSO, 50% FCS, 40% RPMI 1640 medium.

The engineered cells may be infused to a subject in any physiologically acceptable medium by any convenient route of administration, normally intravascularly, although they may also be introduced by other routes, where the cells may find an appropriate site for growth. Usually, at least 1×10⁶ cells/kg will be administered, at least 1×10⁷ cells/kg, at least 1×10⁸ cells/kg, at least 1×10⁹ cells/kg, at least 1×10¹⁰ cells/kg, or more, which may be limited by the number of T cells that are obtained during collection and culture.

In some embodiments, a BHLHE40 modified cell is a CAR T cell. The preparation of CAR T cells is achieved by transforming isolated T cells with a nucleic acid sequence encoding a CAR polypeptide described below. The nucleic acid sequences encoding a CAR and a BHLHE40 sequence may each be provided on separate expression vectors, each nucleic acid sequence being operably linked to one or more expression control elements to achieve expression of the CAR and BHLHE40 sequence in the target cell, the vectors being co-transfected into the target cell. Alternatively, the nucleic acid sequences encoding the CAR and the BHLHE40 sequence may each be provided on a single vector each nucleic acid sequence under the control of one or more expression control elements to achieve expression of the associated nucleic acid sequence. Alternatively, both nucleic acid sequences may be under the control of a single promoter with intervening (e.g. T2A or IRES element) or downstream control elements that facilitate co-expression of the two sequences from the vector.

As used herein, the term “CAR” are used to refer to a polypeptide comprising multiple functional domains arranged from amino to carboxy terminus in the sequence: (a) an antigen binding domain (ABD), (b) a transmembrane domain (TM); and (c) one or more cytoplasmic signaling domains (CSDs) wherein the foregoing domains may optionally be linked by one or more spacer domains. The CAR may also further comprise a signal peptide sequence which is conventionally removed during post-translational processing and presentation of the CAR on the cell surface. CARs useful in the practice of the present invention are prepared in accordance with principles well known in the art. See e.g., Eshhaar et al. U.S. Pat. No. 7,741,465 B1 issued Jun. 22, 2010; Sadelain, et al (2013) Cancer Discovery 3(4):388-398; Jensen and Riddell (2015) Current Opinions in Immunology 33:9-15; Gross, et al. (1989) PNAS(USA) 86(24):10024-10028; Curran, et al. (2012) J Gene Med 14(6):405-15. Examples of commercially available CAR T cell products that may be modified to incorporate an BHLHE40sequence of the present invention include axicabtagene ciloleucel (marketed as Yescarta® commercially available from Gilead Pharmaceuticals) and tisagenlecleucel (marketed as Kymriah® commercially available from Novartis).

As used herein, the term antigen binding domain (ABD) refers to a polypeptide that specifically binds to an antigen expressed on the surface of a target cell. The ABD may be any polypeptide that specifically binds to one or more antigens expressed on the surface of a target cell. In certain embodiments, the target cell antigen is a tumor antigen. Non-limiting examples of tumor antigens that may be targeted by a CAR include one or more antigens selected from the group including, but not limited to, the CD19, CD20, CD30, HER2, IL-11Ra, PSCA, NCAM, NY-ESO-1, MUC1, CD123, FLT3, B7-H3, CD33, IL1RAP, CLL1 (CLEC12A)PSA, CEA, VEGF, VEGF-R2, CD22, ROR1, mesothelin, c-Met, Glycolipid F77, FAP, EGFRvIII, MAGE A3, 5T4, WT1, KG2D ligand, a folate receptor (FRa), GD2, PSMA, BCMA, and Wnt1 antigens.

In one embodiment, the ABD is a single chain Fv (ScFv). An ScFv is a polypeptide comprised of the variable regions of the immunoglobulin heavy and light chain of an antibody covalently connected by a peptide linker (Bird, et al. (1988) Science 242:423-426; Huston, et al. (1988) PNAS(USA) 85:5879-5883; S-z Hu, et al. (1996) Cancer Research, 56, 3055-3061. The generation of ScFvs based on monoclonal antibody sequences is well known in the art. See, e.g. The Protein Protocols Handbook, John M. Walker, Ed. (2002) Humana Press Section 150 “Bacterial Expression, Purification and Characterization of Single-Chain Antibodies” Kipriyanov, S. Antibodies used in the preparation of scFvs may be optimized to select for those molecules which possess particular desirable characteristics (e.g. enhanced affinity) through techniques well known in the art such as phage display and directed evolution. In some embodiments, the ABD comprises an anti-CD19 scFv (see e.g., Cooper, et al., U.S. Pat. No. 9,701,758 issued Jul. 11, 2017, in particular the scFv FMC63 described therein), an anti-PSA scFv, an anti-PSMA scFv (see, e.g. Han, et al (2016) Oncotarget 7(37):59471-59481), an anti-BCMA scFv (see, e.g. the scFv antigen binding domains described in Brogdon, et al. U.S. Pat. No. 10,174,095 issued Jan. 8, 2019), an anti-HER2 scFv, an anti-CEA scFv, an anti-EGFR scFv, an anti-EGFRvIII scFv, an anti-NY-ESO-1 scFv, an anti-MAGE scFv, an anti-5T4 scFv, or an anti-Wnt1 scFv. In another embodiment, the ABD is a single domain antibody (also referred to as VHH) derived from anbitodies obtained through immunization of a camelid (e.g. a camel or llama) with a target cell derived antigen, in particular a tumor antigen. See, e.g. Muyldermans, S. (2001) Reviews in Molecular Biotechnology 74: 277-302. Alternatively, the ABD may be generated wholly synthetically through the generation of peptide libraries and isolating compounds having the desired target cell antigen binding properties in substantial accordance with the teachings or Wigler, et al. U.S. Pat. No. 6,303,313 B1 issued Nov. 12, 1999; Knappik, et al., U.S. Pat. No. 6,696,248 B1 issued Feb. 24, 2004, Binz, et al. (2005) Nature Biotechnology 23:1257-1268, and Bradbury, et al. (2011) Nature Biotechnology 29:245-254.

The ABD may have affinity for more than one target antigen. For example, an ABD of the present invention may comprise chimeric bispecific binding members, i.e. have capable of providing for specific binding to a first target cell expressed antigen and a second target cell expressed antigen. Non-limiting examples of chimeric bispecific binding members include bispecific antibodies, bispecific conjugated monoclonal antibodies (mab)₂, bispecific antibody fragments (e.g., F(ab)₂, bispecific scFv, bispecific diabodies, single chain bispecific diabodies, etc.), bispecific T cell engagers (BiTE), bispecific conjugated single domain antibodies, micabodies and mutants thereof, and the like. Non-limiting examples of chimeric bispecific binding members also include those chimeric bispecific agents described in Kontermann (2012) MAbs. 4(2): 182-197; Stamova et al. (2012) Antibodies, 1(2), 172-198; Farhadfar et al. (2016) Leuk Res. 49:13-21; Benjamin et al. Ther Adv Hematol. (2016) 7(3):142-56; Kiefer et al. Immunol Rev. (2016) 270(1):178-92; Fan et al. (2015) J Hematol Oncol. 8:130; May et al. (2016) Am J Health Syst Pharm. 73(1):e6-e13. In some embodiments, the chimeric bispecific binding member is a bivalent single chain polypeptides. See, e.g. Thirion, et al. (1996) European J. of Cancer Prevention 5(6):507-511; DeKruif and Logenberg (1996) J. Biol. Chem 271(13)7630-7634; and Kay, et al. United States Patent Application Publication Number 2015/0315566 published Nov. 5, 2015. In some instances, a chimeric bispecific binding member may be a bispecific T cell engager (BiTE). A BiTE is generally made by fusing a specific binding member (e.g., a scFv) that binds an antigen to a specific binding member (e.g., a scFv) with a second binding domain specific for a T cell molecule such as CD3. In some instances, a chimeric bispecific binding member may be a CAR T cell adapter. As used herein, by “CAR T cell adapter” is meant an expressed bispecific polypeptide that binds the antigen recognition domain of a CAR and redirects the CAR to a second antigen. Generally, a CAR T cell adapter will have to binding regions, one specific for an epitope on the CAR to which it is directed and a second epitope directed to a binding partner which, when bound, transduces the binding signal activating the CAR. Useful CAR T cell adapters include but are not limited to e.g., those described in Kim et al. (2015) J Am Chem Soc. 137(8):2832-5; Ma et al. (2016) Proc Natl Acad Sci U S A. 113(4):E450-8 and Cao et al. (2016) Angew Chem Int Ed Engl. 55(26):7520-4.

In some embodiments, a linker polypeptide molecule is optionally incorporated into the CAR between the antigen binding domain and the transmembrane domain to facilitate antigen binding. Moritz and Groner (1995) Gene Therapy 2(8) 539-546. In one embodiment, the linker is the hinge region from an immunoglobulin, e.g. the hinge from any one of IgG1, IgG2a, IgG2b, IgG3, IgG4, particularly the human protein sequences. Alternatives include the CH2CH3 region of immunoglobulin and portions of CD3. In those instances where the ABD is an scFv, an IgG hinge may be employed. In some embodiments the linker comprises the amino acid sequence (G₄S)ₙ where n is 1, 2, 3, 4, 5, etc., and in some embodiments n is 3.

CARs further comprise a transmembrane (TM) domain joining the ABD (or linker, if employed) to the intracellular cytoplasmic domain of the CAR. The transmembrane domain is comprised of any polypeptide sequence which is thermodynamically stable in a eukaryotic cell membrane. The transmembrane spanning domain may be derived from the transmembrane domain of a naturally occurring membrane spanning protein or may be synthetic. In designing synthetic transmembrane domains, amino acids favoring alpha-helical structures are preferred. Transmembrane domains useful in construction of CARs are comprised of approximately 10, 11, 12, 13, 14, 15, 16, 17, 18, 19, 20, 21, 22, 22, 23, or 24 amino acids favoring the formation having an alpha-helical secondary structure. Amino acids having a to favor alpha-helical conformations are well known in the art. See, e.g. Pace, et. al. (1998) Biophysical Journal 75: 422-427. Amino acids that are particularly favored in alpha helical conformations include methionine, alanine, leucine, glutamate, and lysine. In some embodiments, the CAR transmembrane domain may be derived from the transmembrane domain from type I membrane spanning proteins, such as CD3ζ, CD4, CD8, CD28, etc.

The cytoplasmic domain of the CAR polypeptide comprises one or more intracellular signal domains. In one embodiment, the intracellular signal domains comprise the cytoplasmic sequences of the T cell receptor (TCR) and co-receptors that initiate signal transduction following antigen receptor engagement and functional derivatives and sub-fragments thereof. A cytoplasmic signaling domain, such as those derived from the T cell receptor ζ-chain, is employed as part of the CAR in order to produce stimulatory signals for T lymphocyte proliferation and effector function following engagement of the chimeric receptor with the target antigen. Examples of cytoplasmic signaling domains include but are not limited to the cytoplasmic domain of CD27, the cytoplasmic domain S of CD28, the cytoplasmic domain of CD137 (also referred to as 4-1BB and TNFRSF9), the cytoplasmic domain of CD278 (also referred to as ICOS), p110α, β, or δ catalytic subunit of PI3 kinase, the human CD3 ζ-chain, cytoplasmic domain of CD134 (also referred to as OX40 and TNFRSF4), FcεR1γ and β chains, MB1 (Igα) chain, B29 (Igβ) chain, etc.), CD3 polypeptides (δ, Δ and ε), syk family tyrosine kinases (Syk, ZAP 70, etc.), src family tyrosine kinases (Lck, Fyn, Lyn, etc.) and other molecules involved in T cell transduction, such as CD2, CD5 and CD28.

In some embodiments, the CAR may also provide a co-stimulatory domain. The term “co-stimulatory domain”, refers to a signaling endodomain of a CAR that provides a secondary non-specific activation mechanism through which a primary specific stimulation is propagated. The co-stimulatory domain refers to the portion of the CAR which enhances the proliferation, survival or development of memory cells. Examples of co-stimulation include antigen nonspecific T cell co-stimulation following antigen specific signaling through the T cell receptor and antigen nonspecific B cell co-stimulation following signaling through the antigen-specific B cell receptor. Co-stimulation, e.g., T cell co-stimulation, and the factors involved have been described in Chen & Flies. (2013) Nat Rev Immunol 13(4):227-42. In some embodiments of the present disclosure, the CSD comprises one or more of members of the TNFR superfamily, CD28, CD137 (4-1BB), CD134 (OX40), Dap10, CD27, CD2, CD5, ICAM-1, LFA-1 (CD11a/CD18), Lck, TNFR-I, TNFR-II, Fas, CD30, CD40 or combinations thereof.

CARs are often referred to as first, second, third or fourth generation. The term first-generation CAR refers to a CAR wherein the cytoplasmic domain transmits the signal from antigen binding through only a single signaling domain, for example a signaling domain derived from the high-affinity receptor for IgE FcεRIγ, or the CD3ζ chain. The single signaling domain contains one or three immunoreceptor tyrosine-based activating motif(s) [ITAM(s)] for antigen-dependent T cell activation. The ITAM-based activating signal endows T cells with the ability to lyse the target tumor cells and secret cytokines in response to antigen binding. Second-generation CARs include a co-stimulatory signal in addition to the CD3ζ-domain. Coincidental delivery of the delivered co-stimulatory signal enhances persistence, cytokine secretion and antitumor activity induced by CAR Transduced T cells. The co-stimulatory domain is usually located membrane proximal relative to the CD3ζ domain. Third-generation CARs include a tripartite signaling domain, comprising for example a CD28, a CD3ζ, and a OX40 or 4-1BB signaling region. Fourth generation CARs, or “armored car” CAR T cells are further gene modified to express or block molecules and/or receptors to enhance immune activity.

Exemplary intracellular signaling domains that may be incorporated into the CAR disclosed herein comprise (amino to carboxy): CD3ζ; CD28-41BB-CD3ζ; CD28-CD3ζ; CD28-OX40-CD3ζ; CD28-41BB-CD3ζ; 41BB-CD-28-CD3ζ and 41BB-CD3ζ.

The term CAR includes CAR variants including but not limited split CARs, ON-switch CARS, bispecific or tandem CARs, inhibitory CARs (iCARs) and induced pluripotent stem (iPS) CAR T cells.

### Therapeutic Cell Formulations

Methods and compositions are provided for enhancing cellular responses, by engineering cells from a recipient or donor to modulate BHLHE40. As discussed above, the subject methods include a step of obtaining the targeted cells, e.g. T cells, which may be isolated from a biological sample, or may be derived in vitro from a source of progenitor cells. The cells are transduced or transfected or treated with anti-sense oligonucleotides, RNAi, etc., which step can be performed in any suitable culture medium. In some embodiments, a population of cells is obtained from a subject and genetically modified ex vivo to introduce a nucleic acid (e.g. a vector) comprising a nucleic acid sequence encoding BHLHE40 operably linked to one or more expression control sequences functional in the isolated cell; or inactivating expression of BHLHE40, and the genetically modified cell is reintroduced into the subject from which it was obtained.

In some embodiments a population of T cells is engineered or treated in vitro to reduce expression of BHLHE40, and then differentiated to a Tr1 phenotype, e.g. a Tallo10 protocol. The cells may be reintroduced into the subject from which it is obtained.

In some embodiments a therapeutic method is provided, the method comprising introducing into a recipient in need thereof of an in vitro engineered cell population, wherein the cell population has been modified as described herein, and is usually autologous or allogeneic with respect to the recipient.

Engineered T cells, e.g. Tallo10 cells, can be provided in pharmaceutical compositions suitable for therapeutic use, e.g. for human treatment. Therapeutic formulations comprising such cells can be frozen, or prepared for administration with physiologically acceptable carriers, excipients or stabilizers (Remington's Pharmaceutical Sciences 16th edition, Osol, A. Ed. (1980)), in the form of aqueous solutions. The cells will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

Generally at least about 10⁴ engineered cells/kg are administered, at least about 10⁵ engineered cells/kg; at least about 10⁶ engineered cells/kg, at least about 10⁷ engineered cells/kg, at least about 10⁸ engineered cells/kg, or more. For example, typical ranges for the administration of cells for use in the practice of the present invention range from about 1×10⁵ to 5×10⁸ viable cells per kg of subject body weight per course of therapy. Consequently, adjusted for body weight, typical ranges for the administration of viable cells in human subjects ranges from approximately 1×10⁶ to approximately 1×10¹³ viable cells, alternatively from approximately 5×10⁶ to approximately 5×10¹² viable cells, alternatively from approximately 1×10⁷ to approximately 1×10¹² viable cells, alternatively from approximately 5×10⁷ to approximately 1×10¹² viable cells, alternatively from approximately 1×10⁸ to approximately 1×10¹² viable cells, alternatively from approximately 5×10⁸ to approximately 1×10¹² viable cells, alternatively from approximately 1×10⁹ to approximately 1×10¹² viable cells per course of therapy. In one embodiment, the dose of the cells is in the range of 2.5-5×10⁹ viable cells per course of therapy.

A course of therapy may be a single dose or in multiple doses over a period of time. In some embodiments, the cells are administered in a single dose. In some embodiments, the cells are administered in two or more split doses administered over a period of 1, 2, 3, 4, 5, 6, 7, 8, 9, 10, 11, 12, 13, 14, 21, 28, 30, 60, 90, 120 or 180 days. The quantity of engineered cells administered in such split dosing protocols may be the same in each administration or may be provided at different levels. Multi-day dosing protocols over time periods may be provided by the skilled artisan (e.g. physician) monitoring the administration of the cells taking into account the response of the subject to the treatment including adverse effects of the treatment and their modulation as discussed above.

In one embodiment, the present invention provides a method of treating a subject suffering from a disease, disorder or condition amendable to treatment with regulatory T cell therapy by the administration of a suitably engineered T cell population. In one embodiment, the present invention provides for a method of treatment, the method comprising the steps of (a) obtaining a biological sample comprising T cells, e.g. naïve C4+ T cells from the individual; (b) enriching the biological sample for the presence of T cells; (c) downregulating expression of BHLHE40 in the T cells; (d) differentiating the T cells to a Tr1 phenotype ex vivo; (e) administering a pharmaceutically effective amount of the CAR T cells to the mammal.

In one embodiment, the present invention provides a method of treating a subject suffering from a disease, disorder or condition amendable to treatment with CAR T cell therapy (e.g. cancer) by the administration of a suitably engineered T cell population. In one embodiment, the present invention provides for a method of treatment of a mammalian subject suffering from a disease, disorder associated with the presence of an aberrant population of cells (e.g. a tumor) said population of cells characterized by the expression of one or more surface antigens (e.g. tumor antigen(s)), the method comprising the steps of (a) obtaining a biological sample comprising T cells from the individual; (b) enriching the biological sample for the presence of T cells; (c) transfecting the T cells with one or more expression vectors comprising a nucleic acid sequence encoding a CAR and a nucleic acid sequence encoding an BHLHE40, the antigen targeting domain of the CAR being capable of binding to at least one antigen present on the aberrant population of cells; (d) expanding the population of CAR T cells ex vivo; (e) administering a pharmaceutically effective amount of the CAR T cells to the mammal.

The preferred formulation depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In still some other embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

### Therapeutic Methods

In some embodiments the subject compositions, methods and kits are used to enhance a T cell mediated immune response, including regulatory T cell responses and CAR T cell responses. In some embodiments the immune response is directed towards a condition where it is desirable to deplete or regulate target cells, e.g., cancer cells, infected cells, regulation of immune cells, including without limitation immune cells involved in autoimmune disease, immune cells involved in transplantation, undesirable inflammatory responses, enhancing erythropoiesis, enhancing thrombopoiesis, etc. Immune conditions may include, without limitation, autoimmune diseases, graft v host disease, hematopoietic bone marrow transplantation, adoptive cell therapy, tumor infiltrating cell (TIL) therapy, inflammation, graft rejection, and the like.

A significant number of patients with hematologic malignancies need a hematopoietic stem cell transplant (HSCT) to be cured. Only about 50% of these patients have a fully matched donor, the remaining patients will require an HSCT from a mismatched related or unrelated donor. Almost 60% of these mismatched donor HSCTs will result in graft-versus-host disease (GvHD), which can cause significant morbidity and increased non-relapse mortality. GvHD is caused by the donor effector T cells present in the HSC graft that recognize and react against the mismatched patient's tissues. In some embodiments a Tallo10 cell composition modified as disclosed herein, is administered to prevent GvHD and induce graft tolerance in patients receiving mismatched unmanipulated donor HSCT. The cell therapy may comprise a cell preparation from the same donor of the HSCT (T-allo10) containing T regulatory type 1 (Tr1) cells able to suppress allogenic (host-specific) responses, thus decreasing the incidence of GvHD.

In some embodiments the condition is cancer. As used herein, the terms “cancer” (or “cancerous”), “hyperproliferative,” and “neoplastic” to refer to cells having the capacity for autonomous or unregulated growth (e.g., an abnormal state or condition characterized by rapidly proliferating cell growth). Hyperproliferative and neoplastic disease states may be categorized as pathologic (e.g., characterizing or constituting a disease state), or they may be categorized as non-pathologic (e.g., as a deviation from normal but not associated with a disease state). The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair. The terms “cancer” or “neoplasm” are used to refer to malignancies of the various organ systems, including those affecting the lung, breast, thyroid, lymph glands and lymphoid tissue, gastrointestinal organs, and the genitourinary tract, as well as to adenocarcinomas which are generally considered to include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to hematologic malignancies, malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. An “adenocarcinoma” refers to a carcinoma derived from glandular tissue or in which the tumor cells form recognizable glandular structures.

Examples of tumor cells include but are not limited to AML, ALL, CML, adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, Hodgkin's lymphoma, Kaposi's sarcoma, kidney cancer, laryngeal and hypopharyngeal cancer, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia.

The compositions and method of the present invention may be combined with additional therapeutic agents. For example, when the disease, disorder or condition to be treated is a neoplastic disease (e.g. cancer) the methods may be combined with conventional chemotherapeutic agents or other biological anti-cancer drugs such as checkpoint inhibitors (e.g. PD1 or PDL1 inhibitors) or therapeutic monoclonal antibodies (e.g., Avastin®, Herceptin®).

Examples of chemical agents identified in the art as useful in the treatment of neoplastic disease, include without limitation, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon.

Targeted therapeutics that can be administered in combination may include, without limitation, tyrosine-kinase inhibitors, such as Imatinib mesylate (Gleevec, also known as STI-571), Gefitinib (Iressa, also known as ZD1839), Erlotinib (marketed as Tarceva), Sorafenib (Nexavar), Sunitinib (Sutent), Dasatinib (Sprycel), Lapatinib (Tykerb), Nilotinib (Tasigna), and Bortezomib (Velcade), Jakafi (ruxolitinib); Janus kinase inhibitors, such as tofacitinib; ALK inhibitors, such as crizotinib; BcI-2 inhibitors, such as obatoclax, venclexta, and gossypol; FLT3 inhibitors, such as midostaurin (Rydapt), IDH inhibitors, such as AG-221, PARP inhibitors, such as Iniparib and Olaparib; PI3K inhibitors, such as perifosine; VEGF Receptor 2 inhibitors, such as Apatinib; AN-152 (AEZS-108) doxorubicin linked to [D-Lys(6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; and/or small molecule drug conjugates, such as Vintafolide; serine/threonine kinase inhibitors, such as Temsirolimus (Torisel), Everolimus (Afinitor), Vemurafenib (Zelboraf), Trametinib (Mekinist), and Dabrafenib (Tafinlar).

Examples of biological agents identified in the art as useful in the treatment of neoplastic disease, include without limitation, cytokines or cytokine antagonists such as IL-12, INFα, or anti-epidermal growth factor receptor, radiotherapy, irinotecan; tetrahydrofolate antimetabolites such as pemetrexed; antibodies against tumor antigens, a complex of a monoclonal antibody and toxin, a T cell adjuvant, bone marrow transplant, or antigen presenting cells (e.g., dendritic cell therapy), anti-tumor vaccines, replication competent viruses, signal transduction inhibitors (e.g., Gleevec® or Herceptin®) or an immunomodulator to achieve additive or synergistic suppression of tumor growth, cyclooxygenase-2 (COX-2) inhibitors, steroids, TNF antagonists (e.g., Remicade® and Enbrel®), interferon-β1a (Avonex®), and interferon-β1b (Betaseron®) as well as combinations of one or more of the foregoing as practiced in known chemotherapeutic treatment regimens readily appreciated by the skilled clinician in the art.

Tumor specific monoclonal antibodies that can be administered in combination with an engineered cell may include, without limitation, Rituximab (marketed as MabThera® or Rituxan®), Alemtuzumab, Panitumumab, Ipilimumab (Yervoy®), etc.

In some embodiments the compositions and methods of the present invention may be combined with immune checkpoint therapy. Examples of immune checkpoint therapies include inhibitors of the binding of PD1 to PDL1 and/or PDL2. PD1 to PDL1 and/or PDL2 inhibitors are well known in the art. Examples of commercially available monoclonal antibodies that interfere with the binding of PD1 to PDL1 and/or PDL2 include nivolumab (Opdivo®, BMS-936558, MDX1106, commercially available from BristolMyers Squibb, Princeton NJ), pembrolizumab (Keytruda®MK-3475, lambrolizumab, commercially available from Merck and Company, Kenilworth NJ), and atezolizumab (Tecentriq®, Genentech/Roche, South San Francisco CA). Additional examples of PD1 inhibitory antibodies include but are not limited to durvalumab (MEDI4736, Medimmune/AstraZeneca), pidilizumab (CT-011, CureTech), PDR001 (Novartis), BMS-936559 (MDX1105, Bristol Myers Squibb), and avelumab (MSB0010718C, Merck Serono/Pfizer) and SHR-1210 (Incyte). Additional antibody PD1 pathway inhibitors are described in U.S. Pat. No. 8,217,149 (Genentech, Inc) issued Jul. 10, 2012; U.S. Pat. No. 8,168,757 (Merck Sharp and Dohme Corp.) issued May 1, 2012, U.S. Pat. No. 8,008,449 (Medarex) issued Aug. 30, 2011, U.S. Pat. No. 7,943,743 (Medarex, Inc) issued May 17, 2011. Additionally, small molecule PD1 to PDL1 and/or PDL2 inhibitors are known in the art. See, e.g. Sasikumar, et al as WO2016142833A1 and Sasikumar, et al. WO2016142886A2, BMS-1166 and BMS-1001 (Skalniak, et al (2017) Oncotarget 8(42): 72167-72181).

The invention now being fully described, it will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXAMPLE 1

Here we show the first full transcriptomic characterization of ex vivo sorted human Tr1 cells. From these analyses we have identified that human Tr1 cells differentially express high levels of Basic Helix-Loop-Helix Family Member E40 (BHLHE40). We characterized BHLHE40's role in primary human CD4+ T cells using CRISPR-Cas9 multiplexed sgRNAs to generate BHLHE40 knockout cells, an in vitro Tr1 differentiation protocol to model Tr1 development, and a temporally controlled method for overexpressing transcription factors in CD4+ T cells. Through these tools, we show that BHLHE40 is required for efficient IFNα and IL-4 production and that BHLHE40 regulates the expression of Tr1 surface molecules LAG3 and CD49b. We hypothesize that BHLHE40 is produced to dampen IL-10 expression in a negative feedback loop and that Tr1 cells specifically.

### Results

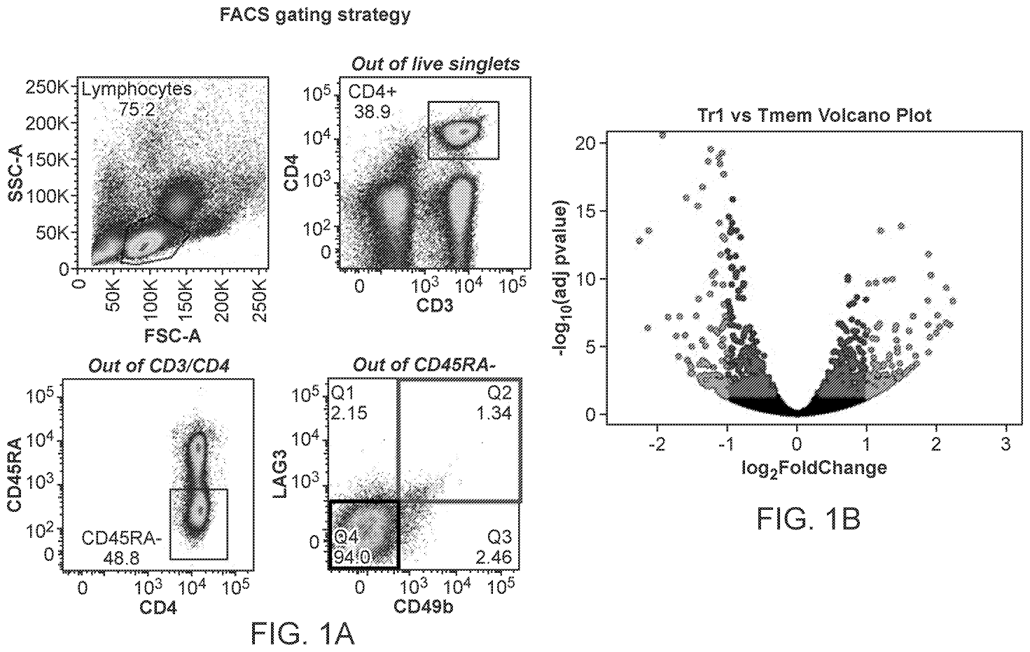

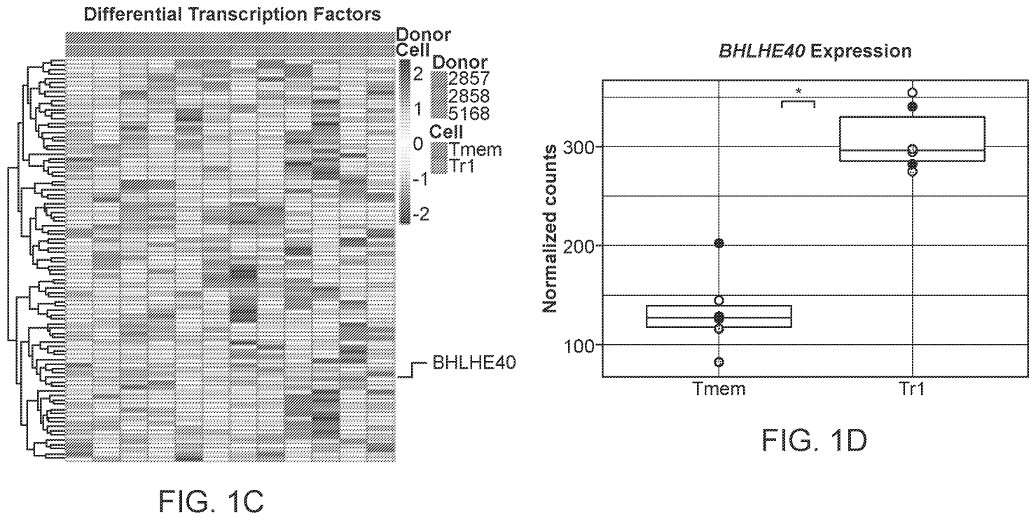

Tr1 cells express high levels of BHLHE40 in vivo. To understand the transcriptome of ex vivo human Tr1 cells in an unbiased, high-throughput approach, we RNA-sequenced sorted Tr1 cells with the phenotype CD3+CD4+CD45RA−LAG3+CD49b+ (FIG. 1A; Q2), and the non-Tr1 memory T cells (Tmem; CD3+CD4+CD45RA−LAG3−CD49b−; FIG. 1A; Q4) directly from the peripheral blood of healthy donors without any in vitro activation. We identified 1,058 genes that were significantly differentially expressed between Tr1 and Tmem cells, with an adjusted p value<0.05 and out of those, 139 genes were overexpressed for >2-fold in Tr1 cells (FIG. 1B; Table 1). We also confirmed differential expression of cMAF, encoded by MAF, which was more highly expressed by Tr1 cells (Table 1). In our effort to identify a master transcription factor in Tr1 cells, analogous to FOXP3+ Tregs, we identified 90 differentially expressed transcription factors with an adjust p value<0.05, among which was the transcription factor BHLHE40 (FIG. 1C). Even though Tr1 cells express extremely high levels of BHLHE40, BHLHE40 is also expressed, albeit at lower levels, in non-Tr1 Tmem (FIG. 1D).

TABLE 1

| | baseMean | log2FoldChange | IfcSE | stat | pvalue | padj |
|---|---|---|---|---|---|---|
| TCF7 | 2744.192 | −0.94488 | 0.113215 | −8.34594 | 7.06E−17 | 2.30E−14 |
| YBX1 | 1252.176 | −1.21679 | 0.167183 | −7.27817 | 3.38E−13 | 6.30E−11 |
| ZNF101 | 334.5128 | −1.41218 | 0.211639 | −6.67259 | 2.51E−11 | 3.28E−09 |
| BHLHE40 | 241.231 | 1.140685 | 0.194792 | 5.855902 | 4.74E−09 | 4.01E−07 |
| GATA3 | 442.5088 | 1.007397 | 0.175544 | 5.738716 | 9.54E−09 | 7.32E−07 |
| FOS | 1313.111 | 0.832361 | 0.150819 | 5.518935 | 3.41E−08 | 2.28E−06 |
| KLF8 | 75.07205 | 1.422065 | 0.262303 | 5.421463 | 5.91E−08 | 3.65E−06 |
| STAT6 | 249.7774 | −0.92332 | 0.179771 | −5.13612 | 2.80E−07 | 1.52E−05 |
| RCOR1 | 236.9698 | 0.904363 | 0.179613 | 5.035056 | 4.78E−07 | 2.49E−05 |
| ARNTL2 | 21.86543 | 1.878405 | 0.375284 | 5.005283 | 5.58E−07 | 2.85E−05 |
| PRDM1 | 2346.137 | 0.596038 | 0.119723 | 4.978497 | 6.41E−07 | 3.15E−05 |
| CSDE1 | 2927.796 | −0.47489 | 0.097529 | −4.86925 | 1.12E−06 | 5.17E−05 |
| AFF2 | 45.87827 | −1.57148 | 0.333961 | −4.70558 | 2.53E−06 | 0.000107 |
| LEF1 | 1231.742 | −0.62538 | 0.133135 | −4.69737 | 2.64E−06 | 0.00011 |
| NR4A2 | 1219.423 | 0.616795 | 0.131598 | 4.686967 | 2.77E−06 | 0.000115 |
| RORA | 3133.441 | 0.497595 | 0.106777 | 4.660127 | 3.16E−06 | 0.000126 |
| NR3C1 | 1352.201 | 0.760296 | 0.165093 | 4.605271 | 4.12E−06 | 0.00016 |
| MXI1 | 608.8098 | −0.6432 | 0.140129 | −4.59005 | 4.43E−06 | 0.00017 |
| HIVEP2 | 3758.985 | −0.52801 | 0.116066 | −4.54925 | 5.38E−06 | 0.000201 |
| BAZ2A | 595.8746 | 0.65317 | 0.143989 | 4.536245 | 5.73E−06 | 0.000212 |
| FOXP1 | 3246.89 | −0.49449 | 0.10943 | −4.51876 | 6.22E−06 | 0.000227 |
| AHR | 1272.35 | 0.668716 | 0.150553 | 4.441722 | 8.92E−06 | 0.000307 |
| BACH2 | 823.8354 | −0.63708 | 0.143529 | −4.4387 | 9.05E−06 | 0.000311 |
| SMARCC1 | 1617.411 | −0.54828 | 0.123727 | −4.43137 | 9.36E−06 | 0.000317 |
| REL | 1055.25 | 0.567236 | 0.134657 | 4.212451 | 2.53E−05 | 0.000746 |
| STAT4 | 783.1287 | 0.547758 | 0.130486 | 4.197828 | 2.69E−05 | 0.000779 |
| MAF | 554.8859 | 0.553202 | 0.136463 | 4.05387 | 5.04E−05 | 0.001341 |
| MECP2 | 309.9783 | 0.779305 | 0.195541 | 3.985385 | 6.74E−05 | 0.00169 |
| TP63 | 19.94439 | 1.472648 | 0.371213 | 3.967124 | 7.27E−05 | 0.0018 |
| MYBL1 | 629.5763 | 0.509975 | 0.129565 | 3.936051 | 8.28E−05 | 0.002011 |
| MLXIP | 224.0176 | −0.75125 | 0.196556 | −3.82208 | 0.000132 | 0.002863 |
| HLF | 119.1194 | 1.070354 | 0.280246 | 3.819339 | 0.000134 | 0.002881 |
| ZNF844 | 36.89255 | −1.24489 | 0.330736 | −3.764 | 0.000167 | 0.003438 |
| ZNF426 | 66.85737 | −1.15833 | 0.307869 | −3.76243 | 0.000168 | 0.00344 |
| UBTF | 165.572 | −0.79051 | 0.214048 | −3.69314 | 0.000221 | 0.004294 |
| HIVEP1 | 1538.446 | −0.45017 | 0.123365 | −3.64906 | 0.000263 | 0.004996 |
| GTF3A | 503.2209 | −0.55198 | 0.151578 | −3.64158 | 0.000271 | 0.00508 |
| MTA2 | 157.7131 | −0.82785 | 0.233477 | −3.54575 | 0.000392 | 0.006779 |
| STAT5A | 141.885 | −0.84787 | 0.246157 | −3.44443 | 0.000572 | 0.009103 |
| MLX | 40.16337 | −1.1339 | 0.330793 | −3.42781 | 0.000608 | 0.00948 |
| FOSL2 | 353.8802 | 0.556471 | 0.163012 | 3.413673 | 0.000641 | 0.009835 |
| ZBTB7A | 409.5302 | 0.592088 | 0.174675 | 3.389657 | 0.0007 | 0.010574 |
| ATF4 | 469.4246 | −0.58612 | 0.175602 | −3.3378 | 0.000844 | 0.012395 |
| ZFP28 | 79.61004 | −0.91798 | 0.277306 | −3.31036 | 0.000932 | 0.013503 |
| ZSCAN29 | 78.73173 | −0.88378 | 0.26942 | −3.28032 | 0.001037 | 0.014677 |
| ZFP62 | 90.30431 | −0.82848 | 0.25393 | −3.26262 | 0.001104 | 0.015363 |
| TERF2 | 122.0275 | −0.80253 | 0.248758 | −3.22614 | 0.001255 | 0.016665 |
| MAX | 556.0661 | −0.46201 | 0.143514 | −3.21928 | 0.001285 | 0.01692 |
| ZNF200 | 25.85705 | −1.11215 | 0.350126 | −3.17641 | 0.001491 | 0.018547 |
| NR4A3 | 134.0353 | 0.761167 | 0.241051 | 3.157701 | 0.00159 | 0.01934 |
| ZHX1 | 195.1954 | −0.61056 | 0.195419 | −3.12434 | 0.001782 | 0.021103 |
| SMAD5 | 224.8278 | 0.700547 | 0.225885 | 3.10135 | 0.001926 | 0.022377 |
| ZNF91 | 714.8718 | −0.45397 | 0.146909 | −3.09016 | 0.002 | 0.023119 |
| IKZF2 | 214.4508 | −0.66456 | 0.215781 | −3.0798 | 0.002071 | 0.023818 |
| POU2F1 | 476.6808 | 0.476603 | 0.154902 | 3.076799 | 0.002092 | 0.023939 |
| TSC22D2 | 165.0389 | 0.692504 | 0.225145 | 3.075814 | 0.002099 | 0.023988 |
| FOSB | 228.8435 | 0.634441 | 0.206404 | 3.073785 | 0.002114 | 0.024068 |
| HMGB1 | 654.8244 | 0.407175 | 0.13247 | 3.073707 | 0.002114 | 0.024068 |
| ZSCAN16 | 22.65207 | −1.08301 | 0.354075 | −3.0587 | 0.002223 | 0.025039 |
| HBP1 | 572.4914 | 0.453429 | 0.14853 | 3.052776 | 0.002267 | 0.0254 |
| SMARCC2 | 560.1928 | −0.4467 | 0.147127 | −3.03615 | 0.002396 | 0.026518 |
| TADA2B | 109.499 | −0.7367 | 0.242797 | −3.03423 | 0.002412 | 0.026623 |
| HMGXB3 | 109.9543 | 0.70293 | 0.232678 | 3.02104 | 0.002519 | 0.027478 |
| KLF11 | 230.2384 | 0.624789 | 0.207128 | 3.016446 | 0.002558 | 0.027699 |
| PRDM2 | 2460.85 | −0.34577 | 0.114857 | −3.01039 | 0.002609 | 0.028191 |
| MAZ | 80.23852 | 0.83689 | 0.279245 | 2.996976 | 0.002727 | 0.029151 |
| ARNTL | 307.4877 | 0.520103 | 0.173614 | 2.99575 | 0.002738 | 0.029234 |
| ZNF687 | 29.45444 | −1.05497 | 0.353123 | −2.98755 | 0.002812 | 0.029856 |
| JUNB | 356.0993 | −0.56143 | 0.189416 | −2.964 | 0.003037 | 0.031686 |
| ETV6 | 634.2925 | 0.482691 | 0.163901 | 2.945004 | 0.00323 | 0.032906 |
| IRF1 | 417.5225 | −0.48315 | 0.164804 | −2.93163 | 0.003372 | 0.033933 |
| ZNF559 | 43.20486 | −0.92372 | 0.318484 | −2.90038 | 0.003727 | 0.03659 |
| NR3C2 | 322.3897 | −0.45681 | 0.158244 | −2.88672 | 0.003893 | 0.037769 |
| TSHZ1 | 186.773 | −0.53119 | 0.186925 | −2.84174 | 0.004487 | 0.041759 |
| PLAG1 | 132.0273 | −0.79546 | 0.280103 | −2.83988 | 0.004513 | 0.041875 |
| NFYC | 153.6648 | −0.59842 | 0.214782 | −2.7862 | 0.005333 | 0.046999 |

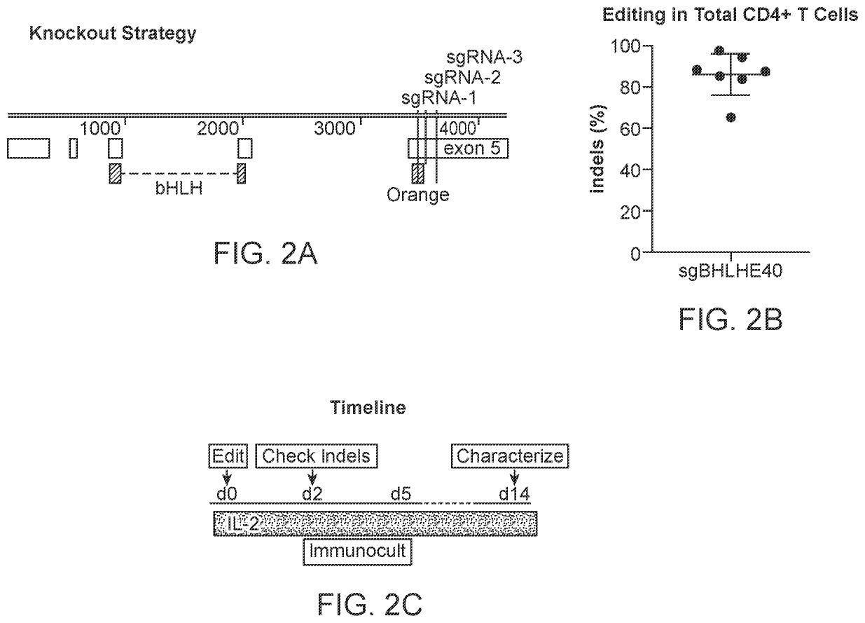

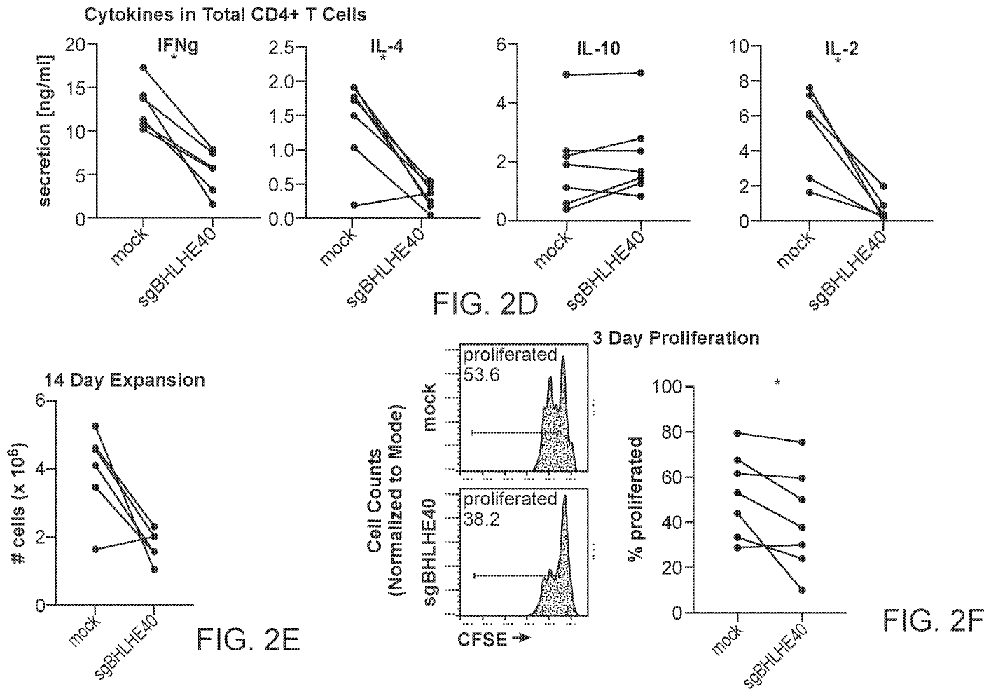

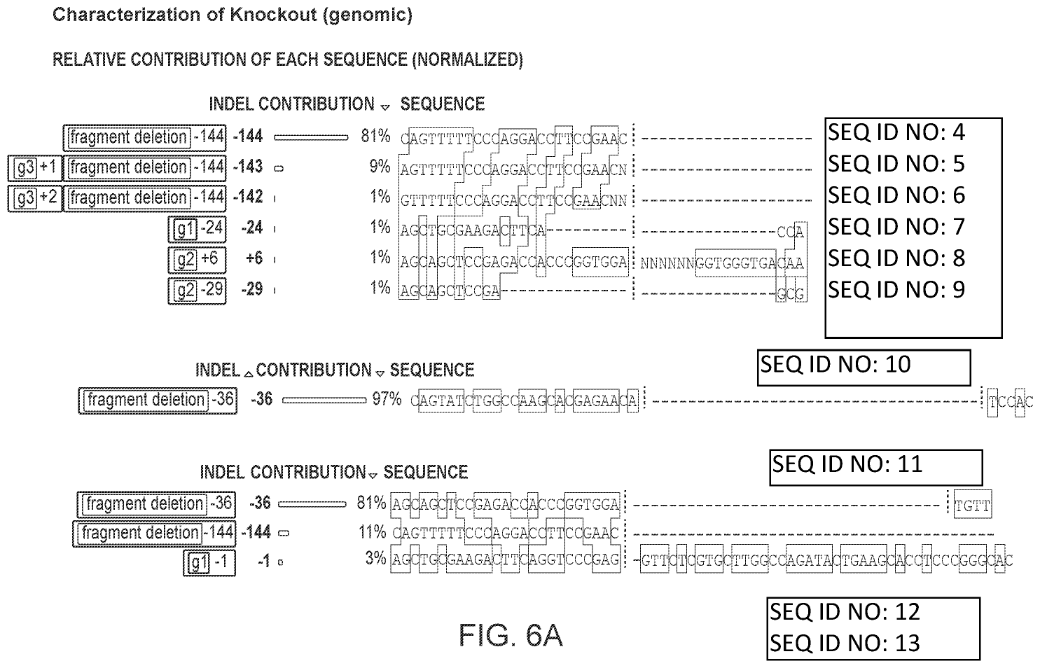

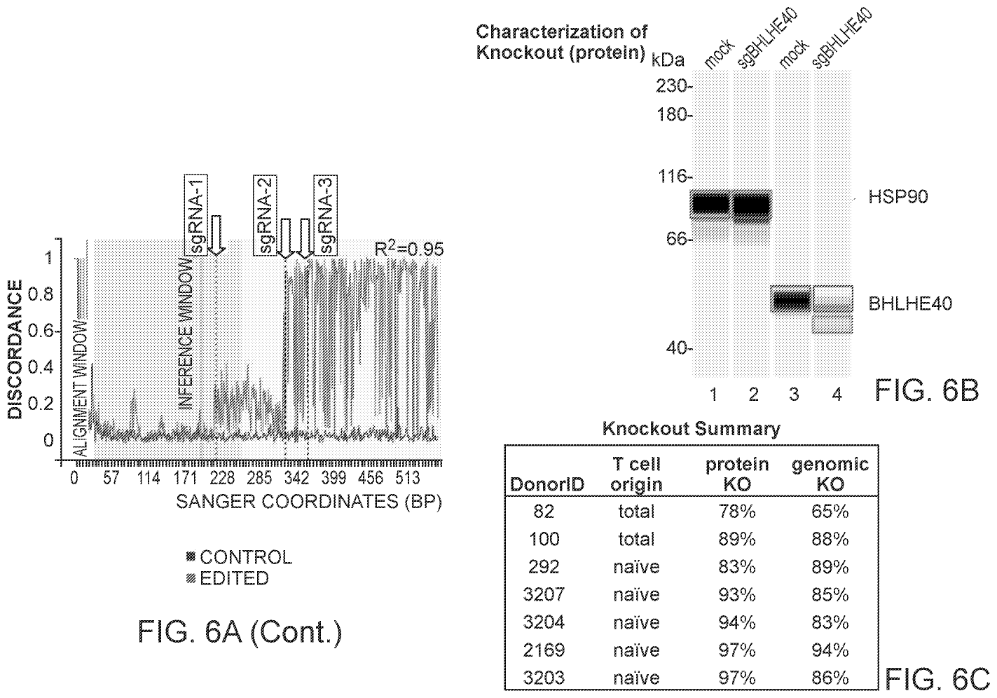

BHLHE40 deficiency reduces IFNγ and IL-4 in total CD4+ T cells. Because the LAG3+CD49b+ Tr1 population represent a small proportion of CD4+ T cells, we first sought to determine the effect of BHLHE40 on total human CD4+ T cells. We generated BHLHE40 mutant CD4+ T cells from healthy donors by nucleofecting three different single guide RNA (sgBHLHE40) multiplexed (FIG. 2A) that were designed to have minimal off target effects and high predicted activity. The multiplexed strategy was additionally designed to cause large deletions that target exon 5, which contains the orange motif of the BHLH-orange domain that is required for DNA binding (FIG. 2A). We disrupted the BHLHE40 locus efficiently: 85.7% insertions and deletions on average (FIG. 2B), with most of the mutants harboring a 36-base pair (bp) or a 144 bp genomic deletion (FIG. 6A). We also confirmed on the protein level that there was a 96% reduction in total protein at the wild type length, some of which could contain the 36 bp deleted-mutant BHLHE40 that leads to only a minor 1.4 kDa shift which cannot be visually resolved, and some truncated protein (FIG. 6B-C).

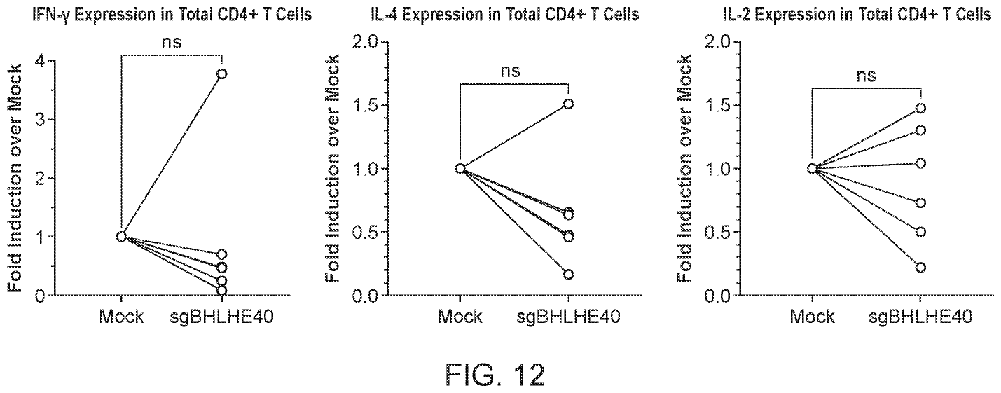

After confirming efficient editing, we expanded them before characterizing their functionality (FIG. 2C). Because ΔBhlhe40 was shown in mouse in vitro differentiated Th1 to be required for optimal IFNγ production and repressed IL-10 over-production (Yu et al., 2018), we first checked if our edited cells exhibited a similar trend. After restimulating our human CD4+ T cells with CD3/CD28, we observed a significant decrease in IFNγ secretion and IL-4 secretion in the BHLHE40 mutant cells (FIG. 2D). We did not detect a significant difference in IL-10 production. When we measured cytokine expression on the transcriptional level, we did not see a significant difference between the wild type and BHLHE40 mutant cells (FIG. 12). Our data suggest that BHLHE40 is required for optimal production of IFNγ and IL-4, which may be reflective of BHLHE40's impact on differentiation of CD4+ T cells into different cell subsets. We also observed a significant decrease in IL-2 secretion after 24 h of CD3/CD28 stimulation in BHLHE40 mutant cells (FIG. 2D), which is consistent with BHLHE40 mutant cells having a reduction in long-term cell expansion in vitro (FIG. 2E) and a decreased rate of cell division after CD3/CD28 stimulation (FIG. 2F).

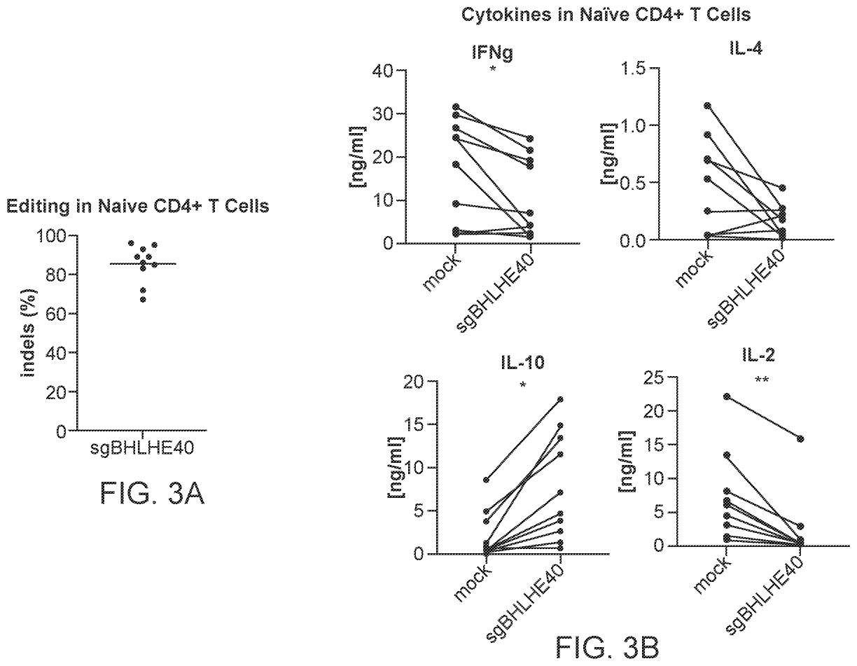

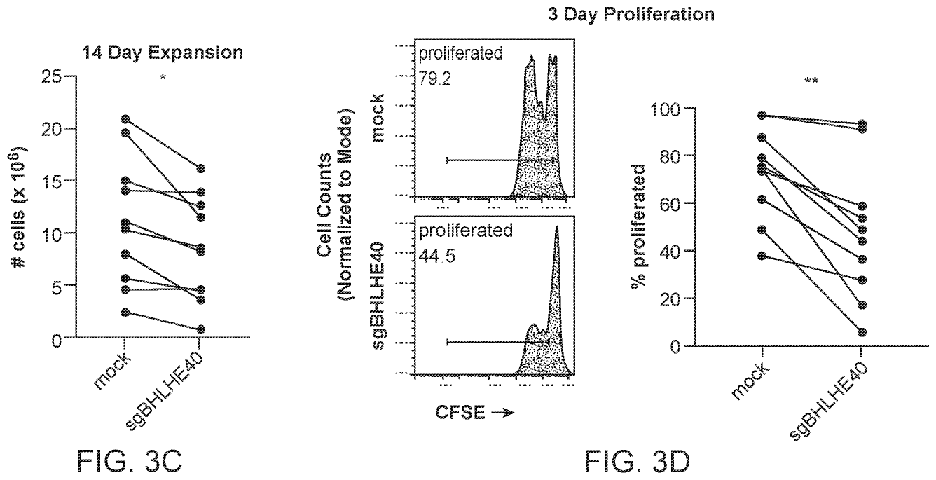

BHLHE40 deficiency increases IL-10 in naïve CD4+ T cells. Knocking out BHLHE40 in total CD4+ T cells provided us with an understanding of BHLEH40's role in maintaining polyfunctionality, such as cytokine production and growth, but we wanted to also assess if it has a distinct role in regulating T cell differentiation. We first isolated naïve CD45RA+CD4+ T cells from both the umbilical cord blood and peripheral blood. Editing in naïve CD4+ T cells was also very efficient and on average we achieved 85.5% indels (FIG. 3A). When we measured cytokine secretion after CD3/CD28 stimulation, as we did for the total CD4+ T cells, we also observed a decrease in IFNγ and IL-2 in BHLHE40 knockout cells (FIG. 3B). While IL-4 levels were lower in BHLHE40 knockout cells, the reduction was not significant, most likely due to the mock cells having overall lower baseline IL-4 secretion (FIG. 3B). On the transcriptionally level, the cytokine expression of IL-10, IL-4, and IFNγ was unchanged after knocking out BHLHE40 (Supplementay FIG. 6). Unlike the edited total CD4+ T cells, the edited naïve CD4+ T cells had an increase in IL-10 secretion, suggesting that BHLHE40's contribution in regulating IL-10 may be more prevalent in the primary response downstream of TCR activation. The increase in IL-10 and decrease in IL-2 in the BHLHE40 knockout cells was observed along with a reduction of cell recovery after in vitro cell expansion (FIG. 3C) and a decrease in cell divisions, indicated by CFSE dilution, immediately following CD3/CD28 stimulation (FIG. 3D).

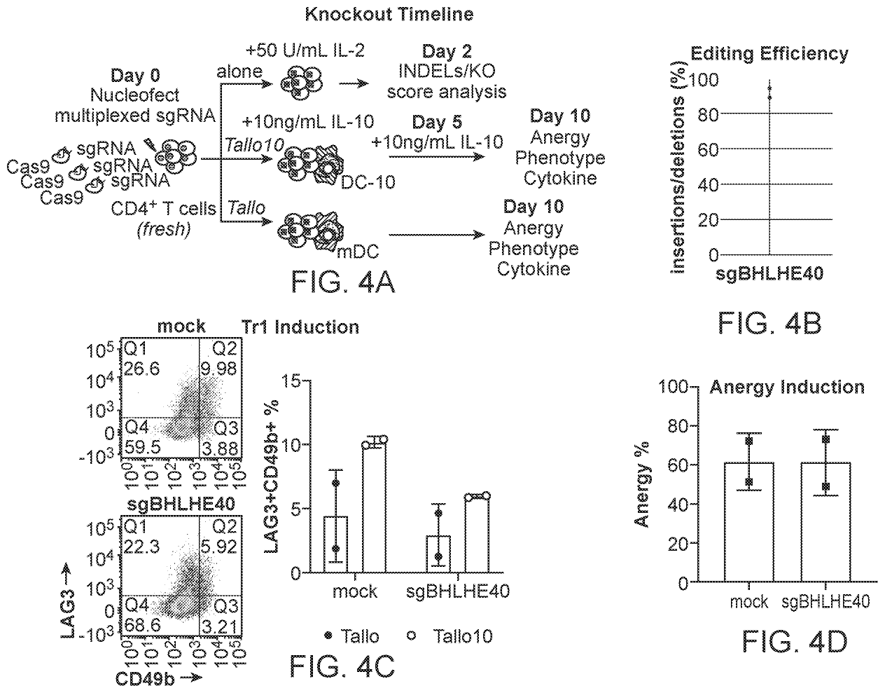

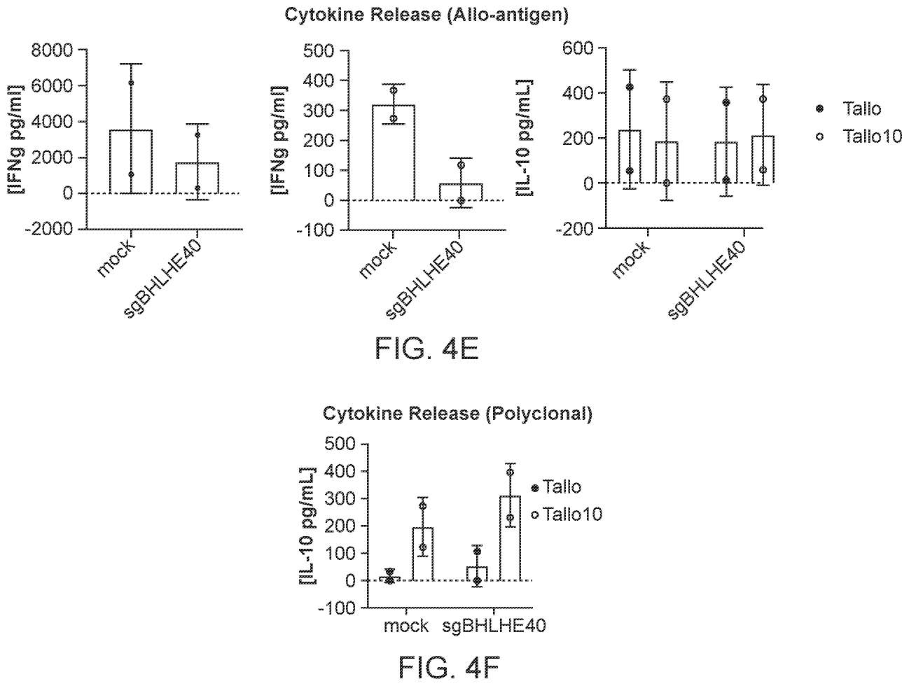

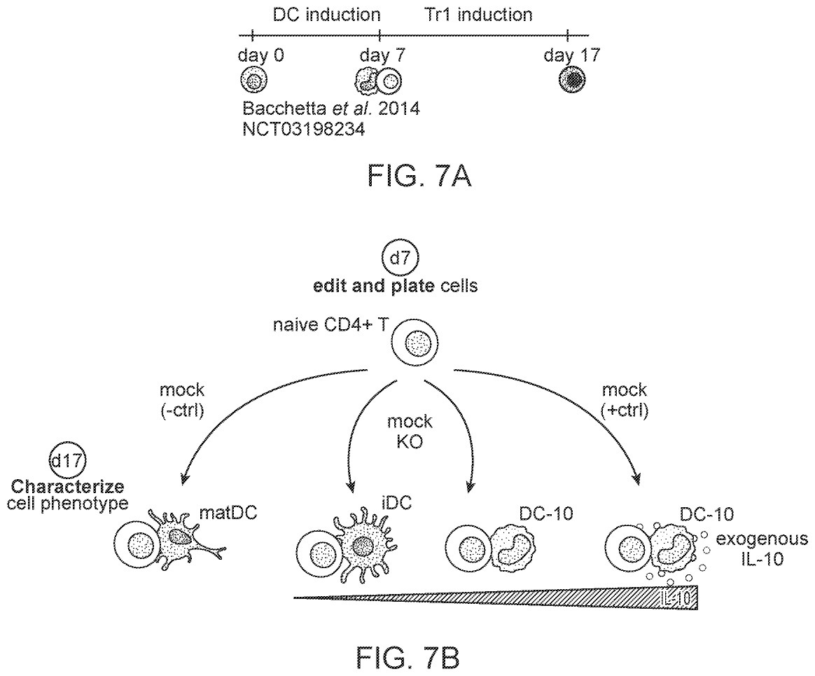

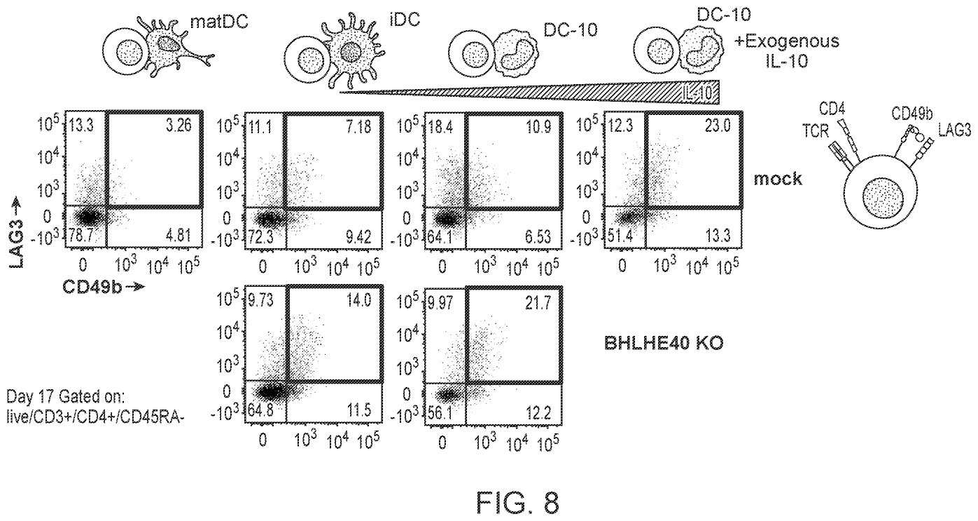

When we differentiated naïve BHLHE40 knockout cells under neutral conditions they produce more IL-10. Therefore, we hypothesized that naïve BHLHE40 knockout cells are poised to differentiate into Tr1 cells more robustly. We combined our protocol for generating BHLHE40 mutants with an establish in vitro Tr1 cell differentiation model in order to specifically investigate the contribution of BHLHE40 in the development of Tr1 cells. This method is a mixed lymphocyte reaction that contains: allogenic-tolerogenic dendritic cells, referred to as DC-10 (Gregori et al., 2010), CD4+ T cells, and IL-10 (Bacchetta et al., 2010), which induces allo-antigen specific anergic T cells, called Tallo10. These Tallo10 cells are currently under clinical investigation (ClinicalTrial.gov ID: NCT03198234). We therefore knocked out BHLHE40 in naïve CD4+ T cells and then plated them with either allogenic DC-10, allogenic mature myeloid dendritic cells (matDC), or alone (FIG. 4A). The cells plated alone enabled us to confirm efficient editing at the BHLHE40 locus without any potential contamination of DC gDNA (FIG. 4B). When Tallo10 induction is perform on non-edited cells in the presence of DC-10 and exogenous IL-10, the average conversion efficiency, as measured by the percentage of phenotypic Tr1s using the markers LAG3 and CD49b is 9-13%. We sought to determine if endogenous IL-10 production in the BHLHE40 knockout cells could enhance the Tr1 conversion efficiency in the absence of exogenous IL-10. Indeed, we saw roughly a 10% increase in phenotypic Tr1 at the end of the Tallo10 induction (FIG. 4C). The increase in PD-1, an important molecule for Tallo10 cell suppression, was also higher in the BHLHE40 knockout cells, which is consistent with the increased Tr1 (FIG. 7A).

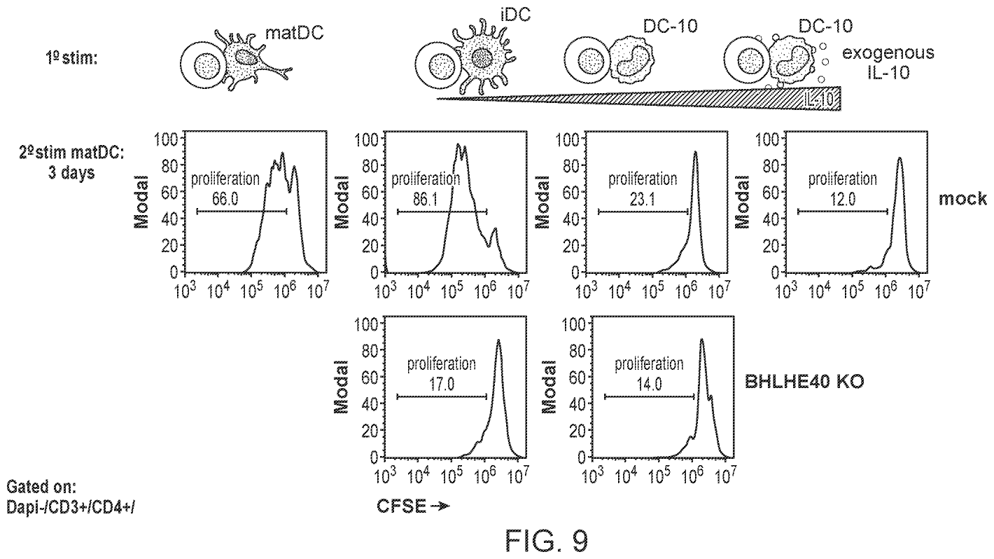

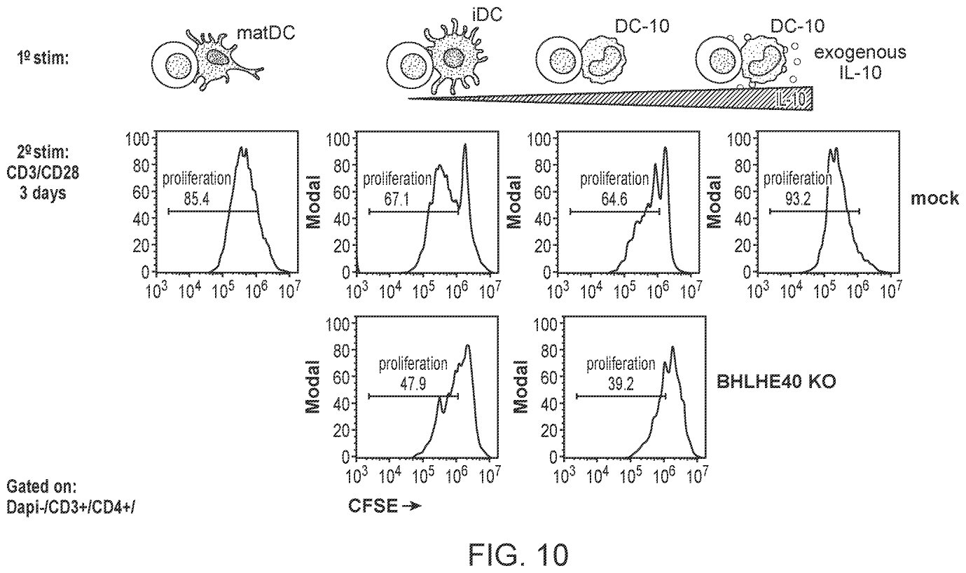

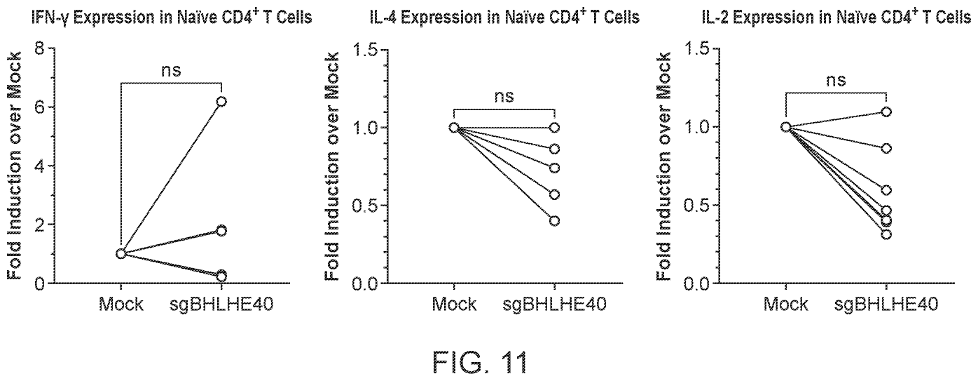

To assess if this increased Tr1 conversion rate in BHLHE40 knockout cells was also met with an increase in allo-antigen specific anergy, we restimulated the cells at the end of the 10-day induction with mDC derived from the same donor as the DC-10. As shown in FIG. 9, BHLHE40 KO cultured with iDC and DC10 are more hyporesponsive to allogenic antigen.

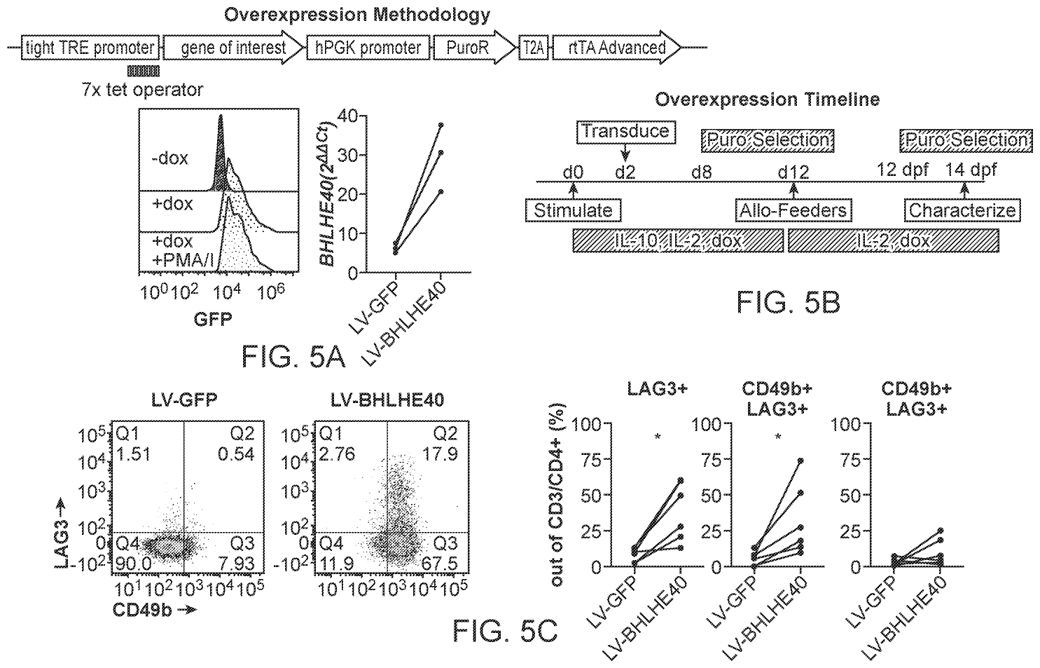

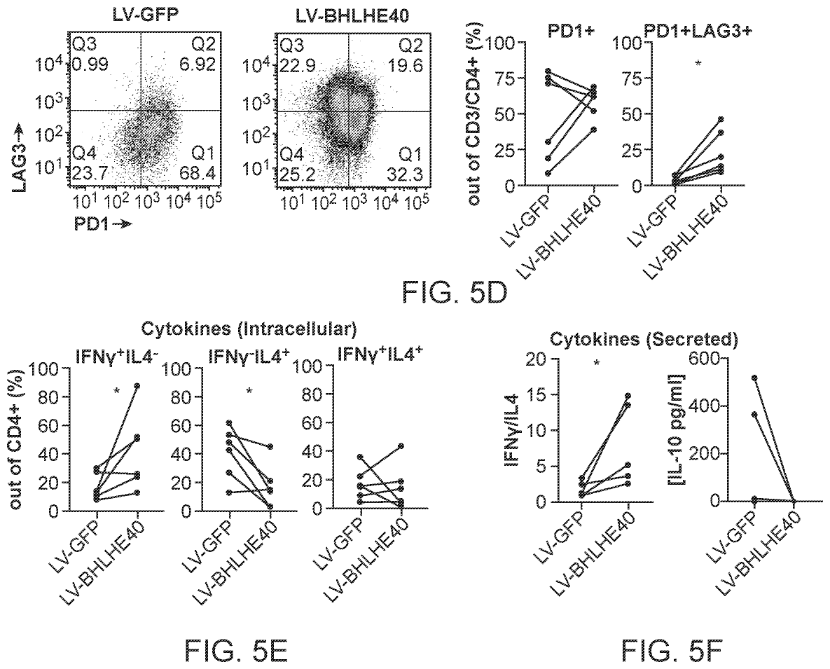

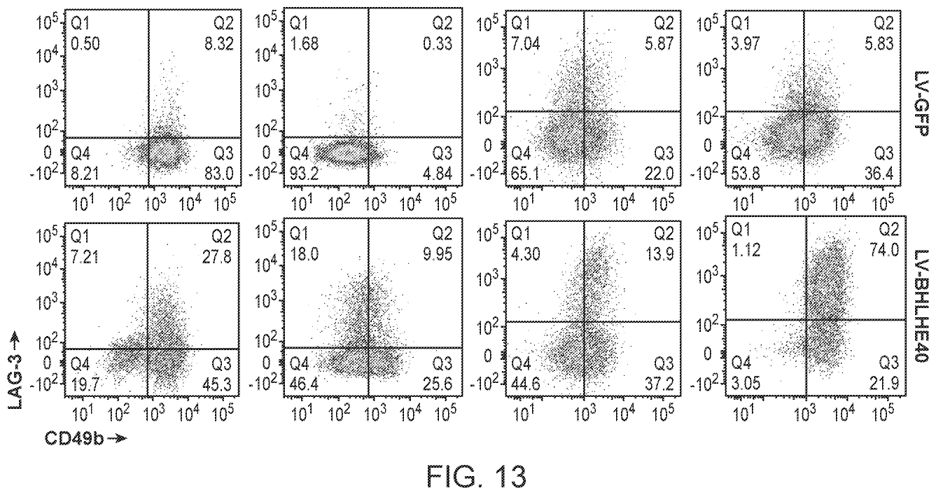

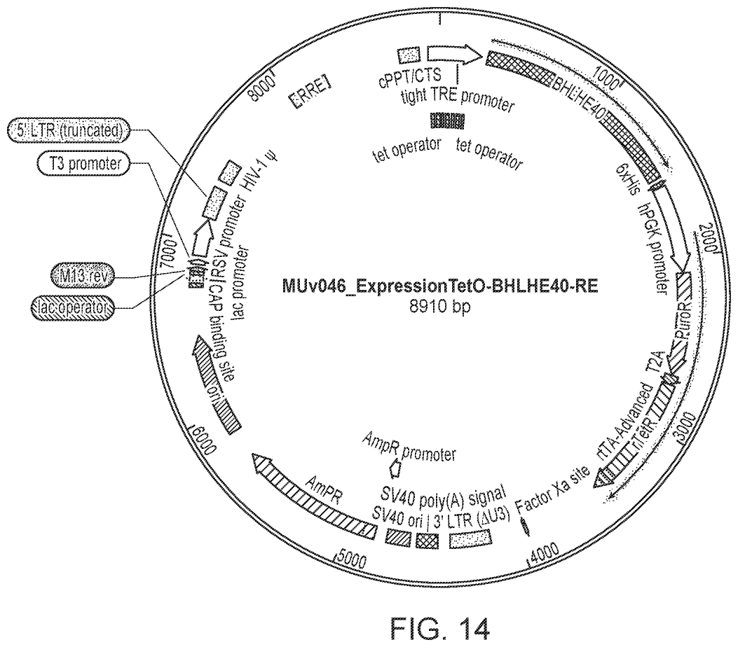

BHLHE40 overexpression increases co-expression of LAG3/CD49b. To explore more deeply the role of BHLHE40 in Tr1 cells, we investigated if overexpression of transcription factor regulates cytokine production and expression of LAG3, CD49b in T cells. BHLHE40 was shown in human naïve T cells to be an important early activated transcription factor downstream of CD28, therefore we started with naïve CD4+ T cells from umbilical cord blood to maximize the window of BHLHE40 influence. We utilized a doxycycline (dox) inducible lentiviral system to enable temporal control of BLHLE40 and control, GFP, expression (FIG. 5A; top). We validated transgene expression in CD4+ T cells after 4 hours of dox exposure and confirmed that expression was constant even during T cells activation (FIG. 5A; bottom-left). Even though BHLHE40 is endogenously expressed, we confirmed dox-inducible expression of BHLHE40 at the RNA level by collected RNA for qPCR after 4 hours of dox exposure in GFP and BHLHE40 transduced cells (FIG. 5A; bottom-right). After validating the overexpression system, we designed a workflow for generating and expanding a pure population of dox responsive cells (FIG. 5B). We first stained the BHLHE40 overexpressing cells and GFP cells for Tr1 surface markers (FIG. 5C; left panel) and found that overexpressing BHLHE40 led to a positive increase in in LAG3+/CD49b+ compared to the GFP controls (FIG. 5C; right panel, FIG. 13). We additionally stained the BHLHE40 overexpressing cells and GFP cells for PD-1, a co-inhibitory molecules commonly associated with Tr1 cells (FIG. 5D; left panel) and found that overexpressing BHLHE40 led to a significant increase in PD1+/LAG3+ compared to the GFP controls (FIG. 5C; right panel). To see if BHLHE40 overexpressing cells had the opposite effect on cytokine production as the BHLHE40 mutant cells, we also checked intracellular cytokine expression and cytokine secretion after stimulation. We observed an increase in intracellular Th1 cytokines (IFNγ+/IL-4−) and a decrease in Th2 cytokines (IFNγ−/IL-4+) in BHLHE40 overexpressing cells normalized to control GFP cells (FIG. 5E). We also saw a similar trend in cytokine secretion; the IFNγ/IL-4 ratio is increased (FIG. 5F). We also had no detectable levels of IL-10 secretion in the BHLHE40 overexpressing cells, suggesting that BHLHE40 can also repress IL-10 secretion in human naïve CD4+ T cells. The decrease in IL-4 was also present when BHLHE40 was knocked out (FIG. 2D); however, the IL-4 reduction in the BHLHE40 overexpressing is accompanied by an increase in IFNγ, which is a known IL-4 repressor.

### Discussion

Coordinated expression of transcription factors are required for the development and function of T cell subsets, which constantly encounter signals such as cytokines and ligands on antigen presenting cells throughout their lifetime. We found that while memory CD4+ T cells express the transcription factor BHLHE40, Tr1 cells express significantly more BHLHE40, suggesting a unique function when very highly expressed. We identified BHLHE40 by RNA sequencing primary human Tr1 cells from the peripheral blood, identified by co-expression of LAG3 and CD49b, to precisely capture Tr1 cells in the most reflective homeostatic state. By investigating BHLHE40's function in primary human CD4+ T cells, we show that its expression affects the production of cytokines IFNγ, IL-4, IL-2, IL-10, proliferation, and regulates the co-expression of LAG3 and CD49b.

Past efforts to better characterize the transcriptome of human Tr1 cells were microarrays of stimulated and unstimulated human Tr1 clones and bulk RNA-sequencing of stimulated human IL-10 producing CD4+ T cells. In public datasets, BHLHE40 is expressed in non-IL-10 producing cells, IL-10 producing cells, but not in FOXP3+ Tregs, suggesting that BHLHE40 is a transcriptional regulator that is utilized by many memory CD4+ T subsets excluding FOXP3+ Tregs regulatory cells. While past methodology of utilizing mouse models to investigate transcription factors in immune development has been fruitful, there are intrinsic limitations and biological differences that must be delineated. Ultimately by utilizing CRISPR-Cas9 technologies and lentiviral overexpression vectors on primary human cells, we have been able to resolve species specific discrepancies and identify new transcriptional control mechanisms surrounding Tr1 biology.

BHLHE40 tightly regulates multiple cytokines, which we show through knockout and overexpression models. We showed that BHLHE40 is required in human CD4+ T cells for efficient production of IFNγ, and IL-4 expression, as seen in the mouse. IFNγ production was reduced in the knockout models and increased in the overexpression models. IL-4 however, remained downregulated regardless of the genetic manipulation, which could be due to BHLHE40 both directly and indirectly controlling IL-4 expression. Unlike in the mouse, IL-10 production only increased when we knocked out BHLHE40 in naïve and not in total CD4+ T cells. These results suggest that in experienced CD4+ T cells, there may be additional IL-10 suppressors in humans that are able to compensate when BHLHE40 is absent. In addition, IL-2 was consistently downregulated in both the naïve and total CD4+ T cell BHLHE40 KO models. Because mouse chromatin immunoprecipitation of BHLHE40 suggests that BHLHE40 has minimal binding to IL-2, we predict that BHLHE40 more likely negative controls IL-2 production through intermediate regulators. Interestingly the reduction in IL-2 was observed alongside a proliferation defect in BHLHE40 knockout cells.

A proliferation defect was reported previously in stimulated naïve CD4+ T cells from Bhlhe40 germline knockout mice but in naïve CD4+ T cells taken from CD4 conditional Bhlhe40 knockout mice, cell recovery after CD3/CD28 stimulation and cell proliferation by CFSE was unchanged. One explanation for the dissimilarities in the data is that BHLHE40 is expressed at other developmental time points and depending on the mouse genetic model, there are molecular changes that affect the ability of BHLHE40 knockout cells to proliferate. The advantage of our system is that we disrupt the BHLHE40 locus in healthy donors after the T cell compartment is established.

BHLHE40 is expressed by memory T cells and even more highly in Tr1 cells, which makes it a multifunctional target whose expression could be modulated for therapeutic purposes. One strategy delivers a molecule to temporarily inhibit BHLHE40 during the conversion of naïve CD4+ T cell to Tr1. Even though Tr1 cells express multiple co-inhibitory molecules that are typically associated as being T cell exhaustion markers such as LAG3 and PD1, they are persistent cells that are fully functional when taken out of the body. We showed that upon overexpressing BHLHE40 in naïve CD4+ T cells, there is an increase in the IFNγ to IL-4 ratio and no IL-10 production. BHLHE40 is ensuring efficient expression of cytokines, which could provide a protective role against T cell exhaustion.

Altogether we propose that Tr1 cells express BHLHE40 as a self-regulatory mechanism. When naïve CD4+ T cells are exposed to antigen and co-stimulation, they are skewed away from differentiating into Tr1 cells because BHLHE40 is quickly upregulated and starts inhibiting IL-10 production. If IL-10 is present during antigen presentation or if BHLHE40 is absent, naïve cells are skewed to differentiate into Tr1 cells. After the Tr1 differentiation is complete, Tr1 cells will start to express high IL-10 and in turn they start to upregulate BHLHE40 in an attempt to dampen IL-10 by upregulating IFNγ. This cycle creates a negative feedback loop between BHLHE40 and IL-10 with IFNγ as the mediator. Ultimately Tr1 cells express so much IL-10 that BHLHE40 is only able to increase IFNγ, which is why Tr1 cells can produce both IL-10 and various levels of IFNγ.

### Materials and Methods

Primary Human Cells. All cells, unless otherwise noted, were obtained from human peripheral blood mononuclear cells (PBMC) from buffy coats of de-identified healthy donors (Stanford Blood Center, Palo Alto, CA, USA) in accordance with IRB guidelines.

RNA Sequencing. 20,000 Tr1 cells (LiveDead Aqua-/CD3+/CD4+/CD45RA−/LAG3+/CD49b+) and non-Tr1 T memory cells (LiveDead Aqua-/CD3+/CD4+/CD45RA−/LAG3−/CD49b−) were isolated by sorting via FACS directly into Trizol-LS in biological duplicates. Nucleic acid was isolated from the aqueous phase after centrifuging with chloroform and RNA was further isolated through concentrating using linear polyacrylamide solution (Sigma Aldrich) and digesting with RQ1 RNAse-free DNAse (Promega). RNA was then cleaned with RNeasy MinElute Cleanup Kit (Qiagen). RNA integrity was checked using the RNA 6000 Pico kit on a bioanalyzer (Agilent). cDNA prep was performed with Nugen Ovation RNAseq system V2. cDNA was sheared using a sonicator (Covaris). Libraries were generated with NEBNext Ultra DNA for Illumina kit. Libraries were normalized and pooled before paired-end 151 bp sequenced on Illumina NextSeq500.

RNA Sequencing processing and analysis. RNA sequencing (RNA-seq) transcripts were trimmed using Skewer, dual passed aligned using STAR to GRCh38. Tabulated gene counts were inported into DESeq2 to perform exploratory analysis, visualization, and differential gene expression using R (version 3.6.0).

Western immunoassay (Wes). 10⁶ T cells were collected 14 days after stimulation and whole-cell extracts were prepared in RIPA lysis buffer containing a protease inhibitor cocktail (Roche, complete Mini Protease Inhibitor Cocktail) and protein concentrations were determined with the BCA protein assay reagent (Pierce). Samples were run on the Wes (ProteinSimple) according to the manufacturer's protocol. 0.5 ug of protein lysate was loaded per lane and the following antibodies were used: DEC1 (Novus Biologicals, NB1001800SS, 1:50) and HSP90 (Cell Signaling Technology, 4877, 1:1000). Image analysis was conducted using the Compass for SW software.

Lentivirus Production. BHLHE40 cDNA ORF was purchased from Sino Biological and cloned into pCW57.1 using cut sites Nhel and Agel. pCW57.1 was a gift from David Root (Addgene plasmid #41393 ; http://n2t.net/addgene:41393; RRID:Addgene_41393). 293T cells were transfected with pMD2.G, pMDLg/pRRE, pILVV01, and pAdvantage (Promega, Madison, WI, USA) using using TransIT®-LT1 (Mirus Bio, USA) and concentrated by ultracentrifugation as previously described (Andolfi et al., 2012; Locafaro et al., 2017). Titer was estimated by limiting dilution on 293T cells by puromycin resistance after transduction of 3 days followed by 2 days of puromycin selection.

Cell Isolation and Culturing. Naïve CD4+ T cells were isolated from CD34− umbilical cord blood cells collected by the Binns Program for Cord Blood Research Cells or from healthy adult peripheral blood by using the EasySep™ Human Naïve CD4+ T Cell Isolation Kit (STEMCELL Technologies, USA); >95% CD3+/CD4+/CD45RA+ cells were obtained. CD4+ T cells were isolated from PBMC using EasySep™ Human CD4+ T Cell Isolation Kit (STEMCELL Technologies, USA) and >95% CD3/CD4+ T cells were obtained. CD4+ T cells were maintained using a base T cell media of 5% Human AB serum (Milipore-Sigma) in X-VIVO-15 (Lonza) and 50 U/ml IL-2, which was refreshed every 3-4 days in addition to any otherwise noted supplements.

Tallo10. CD14+ cells were isolated from PBMCs using the manufacturer's protocol for CD14+ microbeads (Miltenyl) and differentiated into mature DC or DC-10 as previously described with minor modifications (Gregori et al., 2010) with the following changes: CD14+ cells were cultured in a base media using 10% human AB serum and matDC were differentiated with 5 ug/ml of synthetic monophosphoryl lipid A (InvivoGen) on day 5. Cells were collected on day 7 and irradiated at 6000 rads. CD4+ cells were isolated from a donor allogenic to the CD14+ cell donor used and then co-cultured at a 10:1 ratio (T cell:DC) for 10 days with either matDC without cytokine supplements or DC-10 with 10 ng/ml IL-10 added on day 0 and day 5. Cells were collected after 10 days of co-culture.

Overexpression of BHLHE40 in naïve CD4+ T cells. 10⁶ CD4+CD3+CD45RA+ T cells were stimulated as previous described (Locafaro et al., 2017) with the addition of 10 ng/ml IL-10 (BD Biosciences, USA) and transduced with lentivirus with 4 ug/ml polybrene and 1 ug/ml doxycycline (Mirus Bio, USA). 24 hours later, the media was replaced with 50 U/ml IL-2. 10 ng/ml rh IL-10 was added every 4 days for the first 12 days, 1 ug/ml doxycycline was added every two days. Transduced cells were selected for puromycin resistance after culturing with puromycin 1 ng/ml for 4 days. Cells were expanded using an irradiated allogenic feeder mixture (1:10 ratio of JY cell line to human PBMC with 1 ug/ml soluble CD3).

Knockout of BHLHE40. When using frozen CD4+ T cells, cells were thawed in T cell media overnight with 50 U/ml IL-2 before resuspending in P3 solution (Lonza) while freshly isolated CD4+ T cells were directly resuspended in P3 at 10⁶ cells/20 ul and nucleofected with 0.9 pmol of Hi-Fi Cas9 (Integrated DNA Technologies) and 0.33 pmol of each sgRNA (Synthego) using the 4D-16 well strips using program EO115. No differences were observed in editing efficiency or downstream characterization between fresh and frozen starting T cells. Cells were plated directly into T cell media containing 50 U/ml IL-2 after nucleofection. After 2 days, cells were stimulating using 10 ul/1 ml of cells with Immunocult for T cells (STEMCELL Technologies). After 3 days, media was replaced with T cell media by removing the majority of existing media containing the soluble stimulant. 11 days after activation, cells were collected and further characterized.

Insertion and Deletion (Indel). gDNA was isolated with Quick Extract, following manufacturer's protocol. PCR primers were designed 500 bp surrounding the sgRNA cut sites and used to amplify gDNA for Sanger sequencing. Indels were calculated by comparing edited and mock edited cells using Inference of CRISPR Edits, Synthego's Performance Analysis, ICE Analysis. 2019. V2.0. Synthego; [January 2020]

Cytokine. To measure cytokine secretion upon stimulation, 10⁵ T cells were incubated for 48 h on 96-well round bottomed plates pre-coated with immobilized anti-CD3 (10 μg/mL) and soluble anti-CD28 (1 μg/mL). The levels of secreted IL-4, IL-10, and IFN-γ were determined by ELISA with technical duplicates (BD Biosciences). To measure intracellular cytokine production, 10⁵ T cells were incubated for 5 hours on 96-well round bottomed plate containing Leukocyte Activation Cocktail, with GolgiPlug (BD Biosciences) or 10 ng/ml Brefeldin A (Biolegend) only. Cells were then fixed/permed and stained using Fixation/Permeabilization Solution Kit (BD Biosciences) and read on a flow cytometer.

Proliferation. Cells were labeled with 5 uM Carboxyfluorescein succinimidyl ester (CFSE) for 5 mins at room temperature then washed once with fetal bovine serum and once with PBS before resuspending at 10⁶ cells/mL in T cell media. 50,000 cells were plated in a 96-well round bottomed plate with CD3/CD28 Dynabeads (ThermoFisher Scientific) at the indicated ratio and dye dilution was measured on a flow cytometer on the indicated days.

Anergy. Tallo and Tallo10 cells were labeled with CFSE as described in the Proliferation methods section and plated in a 96 well round bottom plate with the following conditions: alone with 50,000 cells per well, irradiated matDC at a 10:1 ratio (Tallo/10: matDC) with 55,000 total cells per well, and 50,000 cells with 2,500 CD3/CD28 Dynabeads (ThermoFisher Scientific). 3 days later, supernatant was collected for cytokine analyses and cells were stained for CD3, CD4, and LiveDead Aqua (ThermoFisher Scientific) and measured on a flow cytometer to quantify the CFSE dye dilution in the CD4+ live T cell gate. CFSE proliferation gates were set using the Tallo/10 CFSE of the cells plated alone. Anergy was calculated with the following equation: (% proliferated Tallo mock−% proliferated Tallo10 mock or sgBHLHE40)/% proliferated Tallo mock.

qPCR. T cells were washed twice in PBS and then resuspended in 300 ul of RLT buffer containing beta-mercaptoethanol. Rneasy Plus Micro Kit (Qiagen) was used according to the manufacture's guidelines to further isolate RNA. RNA was quantified using Qubit RNA HS Assay Kit (ThermoFisher Scientific). cDNA was generated using SuperScript IV VILO Master Mix (ThermoFisher Scientific). BHLHE40, and housekeeping genes including: RPLPO, GAPDH, and beta-Actin were measured using TaqMan Gene Expression Assays with TaqMan Gene Expression Master Mix (ThermoFisher Scientific) on a 384 well plate with a ABI Relative fold gene expression was calculated using the 2^{-ΔΔCt} method

Flow Cytometry. Fluorescence activated cell sorting was performed using BD FACSAria Special Order Research Product. Other flow cytometry assays that did not require sorting was performed on a Cytoflex (Beckman Coulter) or BD FACSAria. Flow cytometry data analysis was performed using FlowJo v10 (FlowJo, Version 10, Ashland, OR, USA).

### REFERENCES

Abbas, A. K., Benoist, C., Bluestone, J. A., Campbell, D. J., Ghosh, S., Hori, S., Jiang, S., Kuchroo, V. K., Mathis, D., Roncarolo, M. G., Rudensky, A., Sakaguchi, S., Shevach, E. M., Vignali, D. A. A., & Ziegler, S. F. (2013). Regulatory T cells: Recommendations to simplify the nomenclature. In Nature Immunology (Vol. 14, Issue 4, pp. 307-308). Nature Publishing Group. https://doi.org/10.1038/ni.2554

Andolfi, G., Fousteri, G., Rossetti, M., Magnani, C. F., Jofra, T., Locafaro, G., Bondanza, A., Gregori, S., & Roncarolo, M. G. (2012). Enforced IL-10 expression confers type 1 regulatory T cell (Tr1) phenotype and function to human CD4+ T cells. Molecular Therapy, 20(9), 1778-1790. https://doi.org/10.1038/mt.2012.71

Bacchetta, R., Bigler, M., Touraine, J.-L., Parkman, R., Tovo, P.-A., Abrams, J., De, R., Malefyt, W., De Vries, J. E., & Roncarolo, M.-G. (1994). High Levels of Interleukin 10 Production In Vivo Are Associated with Tolerance in SCID Patients Transplanted with HLA Mismatched Hematopoietic Stem Cells.

Bacchetta, R., Gregori, S., Serafini, G., Sartirana, C., Schulz, U., Zino, E., Tomiuk, S., Jansen, U., Ponzoni, M., Paties, C. T., Fleischhauer, K., & Roncarolo, M. G. (2010). Molecular and functional characterization of allogantigen specific anergic T cells suitable for cell therapy. Haematologica, 95(12), 2134-2143. https://doi.org/10.3324/haematol.2010.025825

Boisvert, M., Chetoui, N., Gendron, S., & Aoudjit, F. (2010). Alpha2beta1 integrin is the major collagen-binding integrin expressed on human Th17 cells. European Journal of Immunology, 40(10), 2710-2719. https://doi.org/10.1002/eji.201040307

Bruniquel, D., Borie, N., Hannier, S., & Triebel, F. (1998). Regulation of expression of the human lymphocyte activation gene-3 (LAG-3) molecule, a ligand for MHC class II. Immunogenetics, 48(2), 116-124. https://doi.org/10.1007/s002510050411

Constant, S. L., & Bottomly, K. (1997). INDUCTION OF TH1 AND TH2 CD4+ T CELL RESPONSES: The Alternative Approaches. Annual Review of Immunology, 15(1), 297-322. https://doi.org/10.1146/annurev.immunol.15.1.297

Cook, L., Stahl, M., Han, X., Nazli, A., MacDonald, K. N., Wong, M. Q., Tsai, K., Dizzell, S., Jacobson, K., Bressler, B., Kaushic, C., Vallance, B. A., Steiner, T. S., & Levings, M. K. (2019). Suppressive and Gut Reparative Functions of Human Type 1 T-regulatory Cells. Gastroenterology. https://doi.org/10.1053/J.GASTRO.2019.09.002

Dawson, S. R., Turner, D. L., Weintraub, H., & Parkhurst, S. M. (1995). Specificity for the hairy/enhancer of split basic helix-loop-helix (bHLH) proteins maps outside the bHLH domain and suggests two separable modes of transcriptional repression. Molecular and Cellular Biology, 15(12), 6923-6931. https://doi.org/10.1128/mcb.15.12.6923

Doench, J. G., Fusi, N., Sullender, M., Hegde, M., Vaimberg, E. W., Donovan, K. F., Smith, I., Tothova, Z., Wilen, C., Orchard, R., Virgin, H. W., Listgarten, J., & Root, D. E. (2016). Optimized sgRNA design to maximize activity and minimize off-target effects of CRISPR-Cas9. Nature Biotechnology, 34(2), 184-191. https://doi.org/10.1038/nbt.3437

Fontenot, J. D., Gavin, M. A., & Rudensky, A. Y. (2003). Foxp3 programs the development and function of CD4+CD25+ regulatory T cells. Nature Immunology, 4(4), 330-336. https://doi.org/10.1038/ni904

Fujimoto, K., Shen, M., Noshiro, M., Matsubara, K., Shingu, S., Honda, K., Yoshida, E., Suardita, K., Matsuda, Y., & Kato, Y. (2001). Molecular cloning and characterization of DEC2, a new member of basic helix-loop-helix proteins. Biochemical and Biophysical Research Communications, 280(1), 164-171. https://doi.org/10.1006/bbrc.2000.4133

Gabryšová, L., Alvarez-Martinez, M., Luisier, R., Cox, L. S., Sodenkamp, J., Hosking, C., Pérez-Mazliah, D., Whicher, C., Kannan, Y., Potempa, K., Wu, X., Bhaw, L., Wende, H., Sieweke, M. H., Elgar, G., Wilson, M., Briscoe, J., Metzis, V., Langhorne, J., . . . O'Garra, A. (2018). C-Maf controls immune responses by regulating disease-specific gene networks and repressing IL-2 in CD4+ T cells article. Nature Immunology, 19(5), 497-507. https://doi.org/10.1038/s41590-018-0083-5

Gagliani, N., Magnani, C. F., Huber, S., Gianolini, M. E., Pala, M., Licona-Limon, P., Guo, B., Herbert, D. R., Bulfone, A., Trentini, F., Di Serio, C., Bacchetta, R., Andreani, M., Brockmann, L., Gregori, S., Flavell, R. A., & Roncarolo, M.-G. (2013). Coexpression of CD49b and LAG-3 identifies human and mouse T regulatory type 1 cells. Nature Medicine, 19(6), 739-746. https://doi.org/10.1038/nm.3179

Gregori, S., Tomasoni, D., Pacciani, V., Scirpoli, M., Battaglia, M., Magnani, C. F., Hauben, E., & Roncarolo, M. G. (2010). Differentiation of type 1 T regulatory cells (Tr1) by tolerogenic DC-10 requires the IL-10-dependent ILT4/HLA-G pathway. Blood, 116(6), 935-944. https://doi.org/10.1182/blood-2009-07-234872

Groux, H., O'Garra, A., Bigler, M., Rouleau, M., Antonenko, S., De Vries, J. E., & Roncarolo, M. G. (1997). A CD4+ T-cell subset inhibits antigen-specific T-cell responses and prevents colitis. Nature, 389(6652), 737-742. https://doi.org/10.1038/39614

Huynh, J. P., Lin, C.-C., Kimmey, J. M., Jarjour, N. N., Schwarzkopf, E. A., Bradstreet, T. R., Shchukina, I., Shpynov, O., Weaver, C. T., Taneja, R., Artyomov, M. N., Edelson, B. T., & Stallings, C. L. (2018). Bhlhe40 is an essential repressor of IL-10 during Mycobacterium tuberculosis infection. The Journal of Experimental Medicine, 215(7), 1823-1838. https://doi.org/10.1084/jem.20171704

Jarjour, N. N., Bradstreet, T. R., Schwarzkopf, E. A., Cook, M. E., Lai, C.-W., Huang, S. C.-C., Taneja, R., Stappenbeck, T. S., Van Dyken, S. J., Urban, J. F., & Edelson, B. T. (2020). BHLHE40 Promotes T H 2 Cell-Mediated Antihelminth Immunity and Reveals Cooperative CSF2RB Family Cytokines. The Journal of Immunology, 204(4), 923-932. https://doi.org/10.4049/jimmunol.1900978

Knust, E., Schrons, H., Grawe, F., & Campos-Ortega, J. A. (1992). Seven genes of the Enhancer of split complex of Drosophila melanogaster encode helix-loop-helix proteins. Genetics, 132(2), 505-518. /pmc/articles/PMC1205152/?report=abstract

Kunicki, M. A., Amaya Hernandez, L. C., Davis, K. L., Bacchetta, R., & Roncarolo, M.-G. (2018). Identity and Diversity of Human Peripheral Th and T Regulatory Cells Defined by Single-Cell Mass Cytometry. The Journal of Immunology, 200(1), 336-346. https://doi.org/10.4049/jimmunol.1701025

Locafaro, G., Andolfi, G., Russo, F., Cesana, L., Spinelli, A., Camisa, B., Ciceri, F., Lombardo, A., Bondanza, A., Roncarolo, M. G., & Gregori, S. (2017). IL-10-Engineered Human CD4(+) Tr1 Cells Eliminate Myeloid Leukemia in an HLA Class I-Dependent Mechanism. Molecular Therapy: The Journal of the American Society of Gene Therapy, 160(0), 3188-3193. https://doi.org/10.1016/j.ymthe.2017.06.029

Martínez-Llordella, M., Esensten, J. H., Bailey-Bucktrout, S. L., Lipsky, R. H., Marini, A., Chen, J., Mughal, M., Mattson, M. P., Taub, D. D., & Bluestone, J. A. (2013). CD28-inducible transcription factor DEC1 is required for efficient autoreactive CD4+ T cell response. Journal of Experimental Medicine, 210(8), 1603-1619. https://doi.org/10.1084/jem.20122387

Masopust, D., Sivula, C. P., & Jameson, S. C. (2017). Of Mice, Dirty Mice, and Men: Using Mice To Understand Human Immunology. The Journal of Immunology, 199(2), 383-388. https://doi.org/10.4049/jimmunol.1700453

Miyazaki, K., Miyazaki, M., Guo, Y., Yamasaki, N., Kanno, M., Honda, Z., Oda, H., Kawamoto, H., & Honda, H. (2010). The Role of the Basic Helix-Loop-Helix Transcription Factor Dec1 in the Regulatory T Cells. The Journal of Immunology, 185(12), 7330-7339. https://doi.org/10.4049/jimmunol.1001381

Ouyang, W., & O'Garra, A. (2019). IL-10 Family Cytokines IL-10 and IL-22: from Basic Science to Clinical Translation. In Immunity (Vol. 50, Issue 4, pp. 871-891). Cell Press. https://doi.org/10.1016/j.immuni.2019.03.020

Saraiva, M., & Garra, A. O. (2010). The regulation of IL-10 production by immune cells. Nature Reviews Immunology, 10(3), 170-181. https://doi.org/10.1038/nri2711

Sun, H., Lu, B., Li, R. Q., Flavell, R. A., & Taneja, R. (2001). Defective T cell activation and autoimmune disorder in Stra13-deficient mice. Nature Immunology, 2(11), 1040-1047. https://doi.org/10.1038/ni721

Xu, J., Yang, Y., Qiu, G., Lal, G., Wu, Z., Levy, D. E., Ochando, J. C., Bromberg, J. S., & Ding, Y. (2009). c-Maf Regulates IL-10 Expression during Th17 Polarization. The Journal of Immunology, 182(10), 6226-6236. https://doi.org/10.4049/jimmunol.0900123

Yu, F., Sharma, S., Jankovic, D., Gurram, R. K., Su, P., Hu, G., Li, R., Rieder, S., Zhao, K., Sun, B., & Zhu, J. (2018). The transcription factor Bhlhe40 is a switch of inflammatory versus antiinflammatory Th1 cell fate determination. The Journal of Experimental Medicine, 215(7), 1813-1821. https://doi.org/10.1084/jem.20170155

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method of altering phenotype or differentiation in a population of T cells, the method comprising:
modulating expression of BHLHE40 in a population of T cells.

2. The method of claim 1, wherein the T cells are human T cells.

3. The method of claim 1, comprising down-regulating BHLHE40 expression in a population of naïve CD4+ T cells, and differentiating the T cells to Tr1 cells in the presence of one or both of IL-10 and DC10 cells.

4. The method of claim 3, wherein the differentiation to Tr1 cells is in vitro.

5. The method of claim 3, wherein down-regulating BHLHE40 expression is transient.

6. The method of claim 3, wherein BHLHE40 is knocked out in the T cell population.

7. The method of claim 4, wherein cells down-regulated for BHLHE40 are differentiated to Tr1 cells in vitro in a mixed lymphocyte reaction that contains: allogenic-tolerogenic dendritic cells, referred to as DC-10, CD4+ T cells, and IL-10, which induces allo-antigen specific anergic T cells.

8. The method of claim 7, wherein the induces allo-antigen specific anergic T cells are administered to an individual for down-regulation of an immune response.

9. The method of claim 3, wherein the Tr1 cells are selected for expression of LAG3 and CD49b.

10. The method of claim 1, wherein a CAR T cell, is engineered to over-express BHLHE40, which cells are reduced in their tendency to exhaustion.

11. An engineered cell population produced by the method of claim 1.

12. A therapeutic method, comprising introducing into a subject in need thereof a therapeutically effective quantity of an engineered cell population of claim 11.
